(19) United States
(12) Patent Application Publication
(10) Pub. No.: US 2006/0183209 A1
(43) Pub. Date: Aug. 17, 2006

(54) Enzymes for biopolymer production

(75) Inventors: Oliver P. Peoples, Arlington, MA (US); Lara L. Madison, Bridgewater, MA (US); Gjalt W. Huisman, San Carlos, CA (US)

Correspondence Address: PATREA L. PABST;PABST PATENT GROUP LLP, 400 COLONY SQUARE, SUITE 1200, ATLANTA, GA, 30361, US

(73) Assignee: Metabolix, Inc.

(21) Appl. No.: 11/355,440

(22) Filed: Feb. 16, 2006

Related U.S. Application Data
(63) Continuation of application No. 09/364,847, filed on Jul. 30, 1999, now abandoned.
(60) Provisional application No. 60/094,674, filed on Jul. 30, 1998.

(51) Int. Cl. C12N 9/02; C07H 21/04; C12N 5/04

(52) U.S. Cl. 435/189; 435/468; 435/419; 536/23.2

(57) ABSTRACT

In order to optimize the flux or flow of carbon intermediates from normal cellular metabolism into PHAs it is desirable to optimize the expression of the enzymes of the PHA biosynthetic pathway. Gene fusions are genetic constructs where two open reading frames have been fused into one and encode hybrid proteins and in some cases bifunctional hybrid enzymes. Linkers may be added to spatially separate the two domains of the hybrid protein. In the case of enzymes which catalyse successive reactions in a pathway, the fusion of two genes results in bringing two enzymatic activities into close proximity to each other. When the product of the first reaction is a substrate for the second one, this new configuration of active sites may result in a faster transfer of the product of the first reaction to the second active site with a potential for increasing the flux through the pathway.

### CROSS-REFERENCE TO RELATED APPLICATIONS

Priority is claimed to U.S. provisional application Ser. No. 60/094,674, filed Jul. 30, 1998.

### BACKGROUND OF THE INVENTION

The present invention is generally in the field of genetically engineered bacterial and plant systems for production of polyhydroxyalkanoates by microorganisms and genetically engineered plants, wherein the enzymes essential for production of the polymers are expressed as fusion proteins having enhanced properties for polymer synthesis.

Numerous microorganisms have the ability to accumulate intracellular reserves of poly[(R)-3-hydroxyalkanoate] polymers or PHAs. PHAs are biodegradable and biocompatible thermoplastic materials with a broad range of industrial and biomedical applications (Williams and Peoples, 1996, CHEMTECH 26, 38-44). In recent years, the PHA biopolymers have emerged from what was originally considered to be a single homopolymer, poly-3-hydroxybutyrate (PHB), into a broad class of polyesters with different monomer compositions and a wide range of physical properties. Over 100 different monomers have been incorporated into the PHA polymers (Steinbüchel and Valentin, 1995, FEMS Microbiol. Lett. 128; 219-228). It has been useful to divide the PHAs into two groups according to the length of their side chains and their biosynthetic pathways. Those with short side chains, such as polyhydroxybutyrate (PHB), a homopolymer of R-3-hydroxybutyric acid units, are semi-crystalline thermoplastics, whereas PHAs with long side chains are more elastomeric.

Biosynthesis of the short side-chain PHAs such as PHB and PHBV proceeds through a sequence of three enzyme catalyzed reactions from the central metabolite acetyl-CoA. In the first step of this pathway, two acetyl-CoA molecules are condensed to acetoacetyl-CoA by a 3-ketoacyl-CoA thiolase. Acetoacetyl-CoA is subsequently reduced to the PHB precursor 3-hydroxybutyryl-CoA by an NADPH dependent reductase. 3-hydroxybutyryl-CoA is then polymerized to PHB which is sequestered by the bacteria as “intracellular inclusion bodies” or granules. The molecular weight of PHB is generally in the order of 10⁴-10⁷ Da. In some bacteria such as Chromatium vinosum the reductase enzyme is active primarily with NADH as co-factor. The synthesis of the PHBV co-polymer proceeds through the same pathway, with the difference being that acetyl-CoA and propionyl-CoA are converted to 3-ketovaleryl-CoA by β-ketothiolase. 3-ketovaleryl-CoA is then converted to 3-hydroxyvaleryl-CoA which is polymerized.

Long side chain PHAs are produced from intermediates of fatty acid β-oxidation or fatty acid biosynthesis pathways. In the case of β-oxidation, the L-isomer of β-hydroxyacyl-CoA is converted to the D-isomer by an epimerase activity present on the multi-enzyme complex encoded by the faoAB genes. Biosynthesis from acetyl-CoA through the fatty acid synthase route produces the L-isomer of β-hydroxyacyl-ACP. Conversion of the ACP to the CoA derivative is catalyzed by the product of the phaG gene (Kruger and Steinbuchel 1998, U.S. Pat. No. 5,750,848).

Enoyl-CoA hydratases have been implicated in PHA biosynthesis in microbes such as Rhodospirillum rubrum and Aeromonas caviae. The biosynthesis of PHB in R. rubrum is believed to proceed through an acetoacetyl-CoA reductase enzyme specific for the L-isomer of 3-hydroxybutyryl-CoA. Conversion of the L to the D form is then catalysed by the action of two enoyl-CoA hydratase activities. In the case of the PHB-co-HX, where X is a C6-C16 hydroxy acid, copolymers which are usually produced from cells grown on fatty acids, a combination of these routes can be responsible for the formation of the different monomeric units. Indeed, analysis of the DNA locus encoding the PHA synthase gene in Aeromonas caviae, which produces the copolymer PHB-co-3-hydroxyhexanoate, identified a gene encoding a D-specific enoyl-CoA hydratase responsible for the production of the D-β-hydroxybutyryl-CoA and D-β-hydroxyhexanoyl-CoA units (Fukui and Doi, 1997, J. Bacteriol. 179: 4821-4830; Fukui et. al., 1998, J. Bacteriol. 180: 667-673).

It is desirable for economic reasons to be able to produce these polymers in transgenic crop species. Methods for achieving this are known. See, for example, U.S. Pat. No. 5,245,023 and U.S. Pat. No. 5,250,430; U.S. Pat. No. 5,502,273; U.S. Pat. No. 5,534,432; U.S. Pat. No. 5,602,321; U.S. Pat. No. 5,610,041; U.S. Pat. No. 5,650,555: U.S. Pat. No. 5,663,063; WO, 9100917, WO 9219747, WO 9302187, WO 9302194 and WO 9412014, Poirier et. al., 1992, Science 256; 520-523, Williams and Peoples, 1996, Chemtech 26, 38-44. In order to achieve this goal, it is necessary to transfer a gene, or genes in the case of a PHA synthase with more than one subunit, encoding a PHA synthase from a microorganism into plant cells and obtain the appropriate level of production of the PHA synthase enzyme. In addition it may be necessary to provide additional PHA biosynthetic genes, eg. a ketoacyl-CoA thiolase, an acetoacetyl-CoA reductase gene, a 4-hydroxybutyryl-CoA transferase gene or other genes encoding enzymes required to synthesize the substrates for the PHA synthase enzymes.

In many cases, it is particularly desirable to control the expression in different plant tissues or organelles. Methods for controlling expression are known to those skilled in the art (Gasser and Fraley, 1989, Science 244; 1293-1299; Gene Transfer to Plants, 1995, Potrykus, I. and Spangenberg, G. eds. Springer-Verlag Berlin Heidelberg New York. and “Transgenic Plants: A Production System for Industrial and Pharmaceutical Proteins”, 1996, Owen, M. R. L. and Pen, J. Eds. John Wiley & Sons Ltd. England). U.S. Pat. No. 5,610,041 describes the route of plastid expression by the previously known technology of adding a leader peptide to direct the protein expressed from the nuclear gene to the plastid. More recent technology enables the direct insertion of foreign genes directly into the plastid chromosome by recombination (Svab et al., 1990, Proc. Natl. Acad. Sci. USA. 87: 8526-8530; McBride et al., 1994, Proc. Natl. Acad. Sci. USA. 91: 7301-7305). The prokaryotic nature of the plastid RNA and protein synthesis machinery also allows for the expression of microbial genes such as for example the phbC, phbA and phbB genes of R. eutropha.

Genetic engineering of bacteria and plants to make products such as polymers which require the coordinated expression and action of multiple enzymes, sequentially on different substrates, may result in low yields, or poor efficiencies, or variations or deviation in the final product.

It is therefore an object of the present invention to provide methods and materials for enhancing production of products of multiple enzymes, such as polymers, and particularly polyhydroxyalkanoates, in bacteria or plants.

### SUMMARY OF THE INVENTION

In order to optimize the flux or flow of carbon intermediates from normal cellular metabolism into PHAs it is desirable to optimize the expression of the enzymes of the PHA biosynthetic pathway. Gene fusions are genetic constructs where two open reading frames have been fused into one. The transcriptional and translational sequences upstream of the first open reading frame direct the synthesis of a single protein with the primary structure that comprises both original open reading frames. Consequently, gene fusions encode hybrid proteins and in some cases bifunctional hybrid enzymes. Individual genes are isolated, for example, by PCR, such that the resulting DNA fragments contain the complete coding region or parts of the coding region of interest. The DNA fragment that encodes the amino-terminal domain of the hybrid protein may contain a translation initiation site and a transcriptional control sequence. The stop codon in the gene encoding the amino-terminal domain needs to be removed from this DNA fragment. The stop codon in the gene encoding the carboxy-terminal domain needs to be retained in the DNA fragment. DNA sequences that are recognized by restriction enzymes may be introduced into the new genes for DNA cloning purposes. Linkers may be added to spatially separate the two domains of the hybrid protein.

In the case of enzymes which catalyse successive reactions in a pathway, the fusion of two genes results in bringing two enzymatic activities into close proximity to each other. When the product of the first reaction is a substrate for the second one, this new configuration of active sites may result in a faster transfer of the product of the first reaction to the second active site with a potential for increasing the flux through the pathway. The configuration of the two catalytic domains in the hybrid in relation to one another, may be altered by providing a linker sequence between them. This linker may be composed of any of the twenty natural amino acids and can be of variable length. The variation in length and composition are important parameters for changing the relative configuration of the individual domains of the hybrid and its enzyme activities.

This technology allows for the direct incorporation of a series of genes encoding a multi-enzyme pathway into a bacteria or plant or plant organelle, for example, the plastid genome. In some cases it may be useful to re-engineer the 5′-untranslated regions of plastid genes which are important for mRNA stability and translation (Hauser et al., 1996. J. Biol. Chem. 271: 1486-1497), remove secondary structure elements, or add elements from highly expressed plastid genes to maximize expression of transgenes encoded by an operon.

Examples demonstrate the expression of active polypeptides encoding multiple enzyme activies. These are homotetrameric enzymes which require the use of cofactors and which interact to synthesize polymer, which have not previously been demonstrated to be expressable as fusion proteins.

### BRIEF DESCRIPTION OF THE DRAWINGS

FIGS. 1A-1H are schematics of gene fusions encoding multiple-enzyme proteins: pTrcAB including beta-ketothiolase (phbA) and acyl-CoA reductase (phbB) (1A); pTrcBA including phbB and phbA (1B); pTrcCP including PHA synthase (phaC) and phasin (phap) (1C); pTrcPC including phap and phaC (1D); pTrcCG including phaC and beta-hydroxyacyl-ACP::coenzyme-A transferase (phbG) (1E); pTrcGC including phbG and phac (1F); pTrcCJ including phaC and enoyl-CoA hydratases (phaJ) (1G); and pTrcJC including phaJ and phac (1H).

FIG. 2 is a schematic of the construction of pTrcAB11, including phbA and phbB, on a single polypeptide with both thiolase and reductase activity.

### DETAILED DESCRIPTION OF THE INVENTION

I. Gene Fusions

In order to optimize the flux or flow of carbon intermediates from normal cellular metabolism into PHAs it is desirable to optimize the expression of the enzymes of the PHA biosynthetic pathway. Gene fusions are genetic constructs where two open reading frames have been fused into one. The transcriptional and translational sequences upstream of the first open reading frame direct the synthesis of a single protein with the primary structure that comprises both original open reading frames. Consequently, gene fusions encode hybrid proteins and in some cases bifunctional hybrid enzymes. Hybrid proteins have been developed for applications such as protein purification (Bülow, L., Eur. J. Biochem. (1987) 163: 443-448; Bülow, L., Biochem. Soc. Symp. (1990) 57: 123-133); Bülow, L., Tibtech. (1991) 9: 226-231), biochemical analyses (Ljungcrantz et al. FEBS Lett. (1990) 275: 91-94; Ljungcrantz et al., Biochemistry (1989) 28: 8786-8792; Bülow, L., Biochem. Soc. Symp. (1990) 57: 123-133); Bülow, L., Tibtech. (1991) 9: 226-231) and metabolic engineering (U.S. Pat. No. 5,420,027; Carlsson, Biotech. Lett. (1992) 14: 439-444; Bülow, L., Biochem. Soc. Symp. (1990) 57: 123-133); Bülow, L., Tibtech. (1991) 9: 226-231; Fisher, Proc Natl. Acad. Sci. U.S.A. (1992) 89: 10817-10821).

Individual genes are isolated, for example, by PCR, such that the resulting DNA fragments contain the complete coding region or parts of the coding region of interest. The DNA fragment that encodes the amino-terminal domain of the hybrid protein may contain a translation initiation site and a transcriptional control sequence. The stop codon in the gene encoding the amino-terminal domain needs to be removed from this DNA fragment. The stop codon in the gene encoding the carboxy-terminal domain needs to be retained in the DNA fragment. DNA sequences that are recognized by restriction enzymes may be introduced into the new genes for DNA cloning purposes. Linkers may be added to spatially separate the two domains of the hybrid protein.

In the case of enzymes which catalyse successive reactions in a pathway, the fusion of two genes results in bringing two enzymatic activities into close proximity to each other. When the product of the first reaction is a substrate for the second one, this new configuration of active sites may result in a faster transfer of the product of the first reaction to the second active site with a potential for increasing the flux through the pathway. The configuration of the two catalytic domains in the hybrid in relation to one another, may be altered by providing a linker sequence between them. This linker may be composed of any of the twenty natural amino acids and can be of variable length. The variation in length and composition are important parameters for changing the relative configuration of the individual domains of the hybrid and its enzyme activities.

Methods exist for improving the utility of PHA biosynthetic fusion enzymes using molecular evolution or “gene-shuffling” techniques (Stemmer, M. P. C. 1994, Nature, 370: 389-391; Stemmer, M. P. C. 1994, Proc. Natl. Acad. Sci., 1994, 91: 10747-10751). Requirements to make this approach work include the mutagenesis techniques, which are usually PCR-based, and a screening technique to identify those mutant enzymes with the desired improved properties.

A. Genes

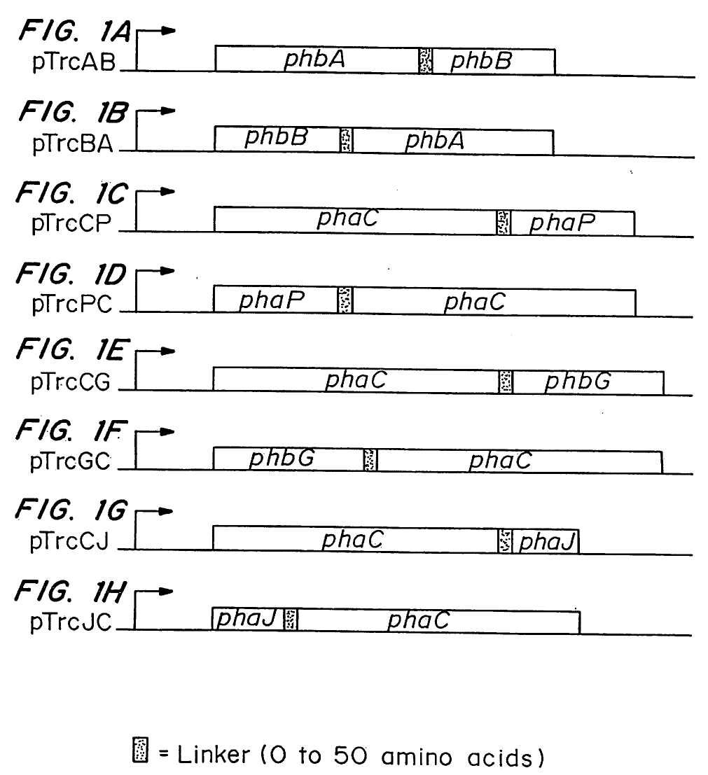

Suitable genes include PHB and PHA synthases, β-ketothiolases, acyl-CoA reductases, phasins, enoyl-CoA hydratases and β-hydroxyacyl-ACP::coenzyme-A transferases. Examples of fusions that can be constructed are illustrated in FIGS. 1A-1H.

β-ketothiolase encoding genes have been isolated from Alcaligenes latus (MBX unpublished; Choi, et al. Appl. Environ. Micrbiol. 64 (12), 4897-4903 (1998)], Ralstonia eutropha [Peoples, O. P. and Sinskey, A. J., J. Biol. Chem. 264: 15298-15303 (1989); Slater et. al., 1998, J. Bacteriol. 180: 1979-1987], Acinetobacter sp. [Schembri, et al. J. Bacteriol., Chromatium vinosum [Liebergesell, M. and Steinbuchel, A. Eur. J. Biochem. 209 (1), 135-150 (1992)], Pseudomonas acidophila (Umeda, et al. Appl. Biochem. Biotech. 70-72: 341-352 (1998)], Pseudomonas denitrificans [Yabutani, et al. FEMS Microbiol. Lett. 133 (1-2), 85-90 (1995)], Rhizobium meliloti [Tombolini, et al. Microbiology 141, 2553-2559 (1995)], Thiocystis violacea [Liebergesell, et al. Appl. Microbiol. Biotechnol. 38 (4), 493-501 (1993)], and Zoogloea ramigera [Peoples, et al. J. Biol. Chem. 262 (1), 97-102 (1987)].

Reductase encoding genes have been isolated from Alcaligenes latus (Choi, et al. Appl. Environ. Micrbiol. 64 (12), 4897-4903 (1998)], R. eutropha [Peoples, O. P. and Sinskey, A. J., J. Biol. Chem. 264 (26), 15298-15303 (1989); Acinetobacter sp. (Schembri, et al. J. Bacteriol), C. vinosum [Liebergesell, M. and Steinbuchel, A. Eur. J. Biochem. 209 (1), 135-150 (1992)], Pseudomonas acidophila (Umeda, et al. Appl. Biochem. Biotech. 70-72: 341-352 (1998)], P. denitrificans [Yabutani, et al. FEMS Microbiol. Lett. 133 (1-2), 85-90 (1995)], R. meliloti [Tombolini, et al. Microbiology 141 (Pt 10), 2553-2559 (1995)], and Z. ramigera [Peoples, O. P. and Sinskey, A. J., 1989, Molecular Microbiology, 3: 349-357).

PHA synthase encoding genes have been isolated from Aeromonas caviae [Fukui, T. and Doi, Y. J. Bacteriol. 179 (15), 4821-4830 (1997)], Alcaligenes latus (Choi, et al. Appl. Environ. Microbiol. 64 (12), 4897-4903 (1998)], R. eutropha [Peoples, O. P. and Sinskey, A. J. J. Biol. Chem. 264 (26), 15298-15303 (1989); Lee, et al. Acinetobacter [Schembri, et al. J. Bacteriol.], C. vinosum [Liebergesell, M. and Steinbuchel, A. Eur. J. Biochem. 209 (1), 135-150 (1992)], Methylobacterium extorquens [Valentin, and Steinbuchel, Appl. Microbiol. Biotechnol. 39 (3), 309-317 (1993)], Nocardia corallina (GenBank Acc. No. AF019964), Nocardia salmonicolor, Pseudomonas acidophila (Umeda, et al. T. Appl. Biochem. Biotech. 70-72: 341-352 (1998)], P. denitrificans [Ueda, et al. J. Bacteriol. 178 (3), 774-779 (1996)], Pseudomonas aeruginosa [Timm, and Steinbuchel, Eur. J. Biochem. 209 (1), 15-30 (1992)], Pseudomonas oleovorans [Huisman, et al. J. Biol. Chem. 266 (4), 2191-2198 (1991)], Rhizobium etli [Cevallos, et al. J. Bacteriol. 178 (6), 1646-1654 (1996)], R. meliloti [Tombolini, et al. Microbiology 141 (Pt 10), 2553-2559 (1995)], Rhodococcus ruber [Pieper, U. and Steinbuechel, A. FEMS Microbiol. Lett. 96 (1), 73-80 (1992)], Rhodospirrilum rubrum [Hustede, et al. FEMS Microbiol. Lett. 93, 285-290 (1992)], Rhodobacter sphaeroides [Steinbüchel, et al. FEMS Microbiol. Rev. 9 (2-4), 217-230 (1992); Hustede, et al. Biotechnol. Lett. 15, 709-714 (1993)], Synechocystis sp. [Kaneko, T., DNA Res. 3 (3), 109-136 (1996)], T. violaceae [Liebergesell, et al. Appl. Microbiol. Biotechnol. 38 (4), 493-501 (1993)], and Z. ramigera (GenBank Acc. No. U66242).

Other genes that have not been implicated in PHA formation but which share significant homology with the phb genes and/or the corresponding gene products may be used as well. Genes encoding thiolase and reductase like enzymes have been identified in a broad range of non-PHB producing bacteria. E. coli (U29581, D90851, D90777), Haemophilus influenzae (U32761), Pseudomonas fragi (D10390), Pseudomonas aeruginosa (U88653), Clostridium acetobutylicum (U08465), Mycobacterium leprae (U00014), Mycobacterium tuberculosis (Z73902), Helicobacter pylori (AE000582), Thermoanaerobacterium thermosaccharolyticum (Z92974), Archaeoglobus fulgidus (AE001021), Fusobacterium nucleatum (U37723), Acinetobacter calcoaceticus (L05770), Bacillus subtilis (D84432, Z99120, U29084) and Synechocystis sp. (D90910) all encode one or more thiolases from their chromosome. Eukaryotic organisms such as Saccharomyces cerevisiae (L20428), Schizosaccharomyces pombe (p89184), Candida tropicalis (D13470), Caenorhabditis elegans (U41105), human (S70154), rat (D13921), mouse (M35797), radish (X78116), pumpkin (D70895) and cucumber (X67696) also express proteins with significant homology to the 3-ketothiolase from R. eutropha.

Genes with significant homology to the phbB gene encoding acetoacetyl CoA reductase have been isolated from several organisms: Azospirillum brasiliense (X64772, X52913) and Rhizobium sp. (U53327, Y00604), E. coli (D90745), Vibrio harveyi (U39441), H. influenzae (U32701), B. subtilis (U59433), P. aeruginosa (U91631), Synechocystis sp. (D90907), H. pylori (AE000570), Arabidopsis thaliana (X64464), Cuphea lanceolata (X64566) and Mycobacterium smegmatis (U66800).

A number of proteins which bind to PHA granules have been identified and their genes cloned (Steinbuchel et. al., 1995, Can. J. Microbiol. (Supplement 1) 41:94-105). The current hypothesis is that these proteins play a role similar to the oleosin oil storage proteins (Huang, A. H. C. 1992, Annu. Rev. Plant Physiol. Plant Mol. Biol. 43: 177-200) in oilseeds and have been named phasins. For example, protein GA24 is a 24 kilodalton protein found in PHA producing cells of Alcaligenes eutrophus (Wieczorek et al., J. Bacteriol. 1995, 177, 2425-2435). The gene encoding GA24, phaP, has been isolated by complementation of PHA-leaky mutants of the bacterium. Wieczorek et al., in their studies of GA24, observed that the protein coated PHA granules in PHA producing cells of A. eutrophus, and that cells deficient in GA24 formed very large granules whereas wild-type cells possessed much smaller granules (Wieczorek et al., J. Bacteriol. 1995, 177, 2425-2435). Based on this observation, the authors proposed that GA24 is one of a number of such proteins termed phasins responsible for controlling PHA granule size. An immunological analysis of other PHA granules from a number of different bacteria indicated conservation of this protein (Wieczorek et. al., 1996, FEMS Microbiology letters 135: 23-30) and the authors concluded that homologs to GA24 are widespread and their genes can be readily isolated. A 13 Kd phasin has been identified in Acinetobacter sp. (Schembri et. al., 1995, FEMS Micro. Lett. 133: 277-283).

B. Transformation Vectors

DNA constructs include transformation vectors capable of introducing transgenes into plants. There are many plant transformation vector options available. See (Gene Transfer to Plants (1995), Potrykus, I. and Spangenberg, G. eds. Springer-Verlag Berlin Heidelberg New York; “Transgenic Plants: A Production System for Industrial and Pharmaceutical Proteins” (1996), Owen, M. R. L. and Pen, J. eds. John Wiley & Sons Ltd. England and Methods in Plant Molecular Biology—a laboratory course manual (1995), Maliga, P., Klessig, D. F., Cashmore, A. R., Gruissem, W. and Vamer, J. E. eds. Cold Spring Laboratory Press, New York).

C. Regulatory Sequences

In general, plant transformation vectors comprise one or more coding sequences of interest under the transcriptional control of 5′ and 3′ regulatory sequences, including a promoter, a transcription termination and/or polyadenylation signal and a selectable or screenable marker gene. The usual requirements for 5′ regulatory sequences include a promoter, a transcription initiation site, and a mRNA processing signal. 3′ regulatory sequences include a transcription termination and/or a polyadenylation signal. Additional RNA processing signals and ribozyme sequences can be engineered into the construct for the expression of two or more polypeptides from a single transcript (U.S. Pat. No. 5,519,164). This approach has the advantage of locating multiple transgenes in a single locus which is advantageous in subsequent plant breeding efforts. An additional approach is to use a vector to specifically transform the plant plastid chromosome by homologous recombination (U.S. Pat. No. 5,545,818), in which case it is possible to take advantage of the prokaryotic nature of the plastid genome and insert a number of transgenes as an operon.

A large number of plant promoters are known and result in either constitutive, or environmentally or developmentally regulated expression of the gene of interest. Plant promoters can be selected to control the expression of the transgene in different plant tissues or organelles, as described by (Gasser and Fraley, 1989, Science 244; 1293-1299). The 5′ end of the transgene may be engineered to include sequences encoding plastid or other subcellular organelle targeting peptides linked in-frame with the transgene. Suitable constitutive plant promoters include the cauliflower mosaic virus 35S promoter (CaMV) and enhanced CAMV promoters (Odell et. al., 1985, Nature, 313: 810), actin promoter (McElroy et al., 1990, Plant Cell 2: 163-171), AdhI promoter (Fromm et. al., 1990, Bio/Technology 8: 833-839; Kyozuka et al., 1991, Mol. Gen. Genet. 228: 40-48), ubiquitin promoters, the Figwort mosaic virus promoter, mannopine synthase promoter, nopaline synthase promoter and octopine synthase promoter. Useful regulatable promoter systems include spinach nitrate-inducible promoter, heat shock promoters, small subunit of ribulose biphosphate carboxylase promoters and chemically inducible promoters (U.S. Pat. No. 5,364,780 and U.S. Pat. No. 5,364,780).

It may be preferable to express the transgenes only in the developing seeds. Promoters suitable for this purpose include the napin gene promoter (U.S. Pat. No. 5,420,034; U.S. Pat. No. 5,608,152), the acetyl-CoA carboxylase promoter (U.S. Pat. No. 5,420,034; U.S. Pat. No. 5,608,152), 2S albumin promoter, seed storage protein promoter, phaseolin promoter (Slightom et. al., 1983, Proc. Natl. Acad. Sci. USA 80: 1897-1901), oleosin promoter (plant et. al., 1994, Plant Mol. Biol. 25: 193-205; Rowley et. al., 1997, Biochim. Biophys. Acta. 1345: 1-4; U.S. Pat. No. 5,650,554; PCT WO 93/20216), zein promoter, glutelin promoter, starch synthase promoter, and starch branching enzyme promoter.

A number of useful plant vectors comprising many of the features described above have been described in the literature. Particularly useful among these are the “super-binary” vectors described by Ishida et. al., (1996, Nature biotechnology 14: 745-750) and the extensive range of vectors available from Cambia, Canberra, Australia (described by Roberts et. al., “A comprehensive set of modular vectors for advanced manipulations and efficient transformation of plants” presented at the Rockefeller Foundation Meeting of the International Program on Rice Biotechnology, 15-18 Sep. 1997, Malacca, Malaysia).

II. Methods for Transformation of Plants and Selection Thereof

It is preferable to express more than one gene product in the plant. A number of methods can be used to achieve this including: introducing the encoding DNAs in a single transformation event where all necessary DNAs are on a single vector; in a co-transformation event where all necessary DNAs are on separate vectors but introduced into plant cells simultaneously; introducing the encoding DNAs by independent transformation events successively into the plant cells i.e. transformation of transgenic plant cells expressing one or more of the encoding DNAs with additional DNA constructs; transformation of each of the required DNA constructs by separate transformation events, obtaining transgenic plants expressing the individual proteins and using traditional plant breeding methods to incorporate the entire pathway into a single plant.

The transformation of suitable agronomic plant hosts using these vectors can be accomplished by a range of methods and plant tissues. Suitable plants include: the Brassica family including napus, rappa, sp. carinata and juncea, maize, soybean, cottonseed, sunflower, palm, coconut, safflower, peanut, mustards including Sinapis alba and flax. Suitable tissues for transformation using these vectors include protoplasts, cells, callus tissue, leaf discs, pollen, meristems etc. Suitable transformation procedures include Agrobacterium-mediated transformation, biolistics, microinjection, electroporation, polyethylene glycol-mediated protoplast transformation, liposome-mediated transformation, silicon fiber-mediated transformation (U.S. Pat. No. 5,464,765) etc. (Gene Transfer to Plants (1995), Potrykus, I. and Spangenberg, G. eds. Springer-Verlag Berlin Heidelberg New York; “Transgenic Plants: A Production System for Industrial and Pharmaceutical Proteins” (1996), Owen, M. R. L. and Pen, J. eds. John Wiley & Sons Ltd. England and Methods in Plant Molecular Biology—a laboratory course manual (1995), Maliga, P., Klessig, D. F., Cashmore, A. R., Gruissem, W. and Varner, J. E. eds. Cold Spring Laboratory Press, New York).

Transformation procedures have been established for these specific crops (Gene Transfer to Plants (1995), Potrykus, I. and Spangenberg, G. eds. Springer-Verlag Berlin Heidelberg New York; “Transgenic Plants: A Production System for Industrial and Pharmaceutical Proteins” (1996), Owen, M. R. L. and Pen, J. eds. John Wiley & Sons Ltd. England and Methods in Plant Molecular Biology-A laboratory course manual (1995), Maliga, P., Klessig, D. F., Cashmore, A. R., Gruissem, W. and Varner, J. E. eds. Cold Spring Laboratory Press, New York).

Brassica napus can be transformed as described for example in U.S. Pat. No. 5,188,958 and U.S. Pat. No. 5,463,174. Other Brassica such as rappa, carinata and juncea as well as Sinapis alba can be transformed as described by Moloney et. al., (1989, Plant Cell Reports 8: 238-242). Soybean can be transformed by a number of reported procedures. See (U.S. Pat. No. 5,015,580; U.S. Pat. No. 5,015,944; U.S. Pat. No. 5,024,944; U.S. Pat. No. 5,322,783; U.S. Pat. No. 5,416,011; U.S. Pat. No. 5,169,770). A number of transformation procedures have been reported for the production of transgenic maize plants including pollen transformation (U.S. Pat. No. 5,629,183), silicon fiber-mediated transformation (U.S. Pat. No. 5,464,765) electroporation of protoplasts (U.S. Pat. No. 5,231,019; U.S. Pat. No. 5,472,869; U.S. Pat. No. 5,384,253) gene gun (U.S. Pat. No. 5,538,877; U.S. Pat. No. 5,538,880 and Agrobacterium-mediated transformation (EP 0 604 662 A1; WO 94/00977). The Agrobacterium-mediated procedure is particularly preferred as single integration events of the transgene constructs are more readily obtained using this procedure which greatly facilitates subsequent plant breeding. Cotton can be transformed by particle bombardment (U.S. Pat. No. 5,004,863; U.S. Pat. No. 5,159,135). Sunflower can be transformed using a combination of particle bombardment and Agrobacterium infection (EP 0 486 233 A2; U.S. Pat. No. 5,030,572). Flax can be transformed by either particle bombardment or Agrobacterium-mediated transformation. Recombinase technologies which are useful in practicing the current invention include the cre-lox, FLP/FRT and Gin systems. Methods by which these technologies can be used for the purpose described herein are described, for example, in U.S. Pat. No. 5,527,695; Dale And Ow, 1991, Proc. Natl. Acad. Sci. USA 88: 10558-10562; Sauer, 1993, Methods in Enzymology 225: 890-900; Medberry et. al., 1995, Nucleic Acids Res. 23: 485-490. U.S. Pat. No. 5,723,764 describes a method for controlling plant gene expression using cre/lox.

Selectable marker genes include the neomycin phosphotransferase gene nptII (U.S. Pat. No. 5,034,322, U.S. Pat. No. 5,530,196), hygromycin resistance gene (U.S. Pat. No. 5,668,298), bar gene encoding resistance to phosphinothricin (U.S. Pat. No. 5,276,268). EP 0 530 129 A1 describes a positive selection system which enables the transformed plants to outgrow the non-transformed lines by expressing a transgene encoding an enzyme that activates an inactive compound added to the growth media. Useful screenable marker genes include the β-glucuronidase gene (Jefferson et. al., 1987, EMBO J. 6: 3901-3907; U.S. Pat. No. 5,268,463) and native or modified green fluorescent protein gene (Cubitt et. al., 1995, Trends Biochem Sci. 20: 448-455; Pang et. al., 1996, Plant Physiol. 112: 893-900). Some of these markers have the added advantage of introducing a trait such as herbicide resistance into the plant of interest providing an additional agronomic value on the input side.

Following transformation by any one of the methods described above, the following procedures can be used to obtain a transformed plant expressing the transgenes of the current invention: select the plant cells that have been transformed on a selective medium; regenerate the plant cells that have been transformed to produce differentiated plants; and select transformed plants expressing the transgene at such that the level of desired polypeptide is obtained in the desired tissue and cellular location.

The examples demonstrate the synthesis of new genetically engineered enzymes for the efficient production of polyhydroxyalkanoate biopolymers in transgenic organisms. In one example, the thiolase and reductase activities encoded by the phbA and phbB genes have been combined into a single enzyme through the construction of a gene fusion. Use of such a hybrid enzyme and its corresponding gene is advantageous: combining two enzyme activities in a single transcriptional unit reduces the number of genes that need to be expressed in transgenic organisms, and the close proximity of two enzyme activities which catalyse sequential steps in a metabolic pathway. On the fusion enzyme allows for direct transfer of the reaction product from the first catalytic domain to the second domain. These gene fusions can be applied in transgenic microbial or plant crop PHA production systems. The fusions can be expressed in the cytosol or subcellular organelles of higher plants such as the seed of an oil crop (Brassica, sunflower, soybean, corn, safflower, flax, palm or coconut), starch accumulating plants (potato, tapioca, cassaya), fiber plants (cotton, hemp) or the green tissue of tobacco, alfalfa, switchgrass or other forage crops.

### EXAMPLES

The present invention will be further understood by reference to the following examples, which use these general methods and materials:

DNA manipulations were performed on plasmid and chromosomal DNA purified with the Qiagen plasmid preparation or Qiagen chromosomal DNA preparation kits according to manufacturers recommendations. DNA was digested using restriction enzymes (New England Biolabs, Beverly, Mass.) according to manufacturers recommendations. DNA fragments were isolated from 0.7% agarose-Tris/acetate/EDTA gels using a Qiagen kit. Oligonucleotides were purchased from Biosynthesis or Genesys. DNA sequences-were determined by automated sequencing using a Perkin-Elmer ABI 373A sequencing machine. DNA was amplified using the polymerase-chain-reaction in 50 microliter volume using PCR-mix from Gibco-BRL (Gaithersburg, Md.) and an Ericomp DNA amplifying machine.

E. coli strains were grown in Luria-Bertani medium or 2xYT medium (Sambrook et. al., 1992, in Molecular Cloning, a laboratory manual, 2nd Ed., Cold Spring Harbor Laboratory Press, Cold Spring Harbor, N.Y.). at 37° C., 30° C. or 16° C.

Accumulated PHB was determined by gas chromatographic (GC) analysis, carried out on the lyophilized cell mass. About 20 mg of lyophilized cell mass was subjected to simultaneous extraction and butanolysis at 110° C. for 3 hours in 2 mL of a mixture containing (by volume) 90% 1-butanol and 10% concentrated hydrochloric acid, with 2 mg/mL benzoic acid added as an internal standard. The water-soluble components of the resulting mixture were removed by extraction with 3 mL water. The organic phase (1 μL at a split ratio of 1:50 at an overall flow rate of 2 mL/min) was analyzed on an HP 5890 GC with FID detector (Hewlett-Packard Co, Palo Alto, Calif.) using an SPB-1 fused silica capillary GC column (30 m; 0.32 mm ID; 0.25 μm film; Supelco; Bellefonte, Pa.) with the following temperature profile: 80° C., 2 min; 10 C.° per min to 250° C.; 250° C., 2 min. Butylbenzoate was used as an internal standard. Molecular weights of the isolated polymers were determined by GPC using a Waters Styragel HT6E column (Millipore Corp., Waters Chromatography Division, Milford, Mass.) calibrated vs. polystyrene samples of narrow polydispersity. Samples were dissolved in chloroform at 1 mg/mL, 50 μL samples were injected and eluted at 1 mL/min. Detection was performed using a differential refractometer.

Protein samples were denatured by incubation in a boiling water bath (3 minutes) in the presence of 2-mercaptoethanol and sodium dodecylsulphate and subsequently separated on 10%, 15% or 10-20% sodium dodecylsulphate-polyacrylamide gels (SDS-PAGE). After transfer of protein to supported nitrocellulose membranes (Gibco-BRL, Gaithersburg, Md.), 3-ketoacyl-CoA thiolase, acetoacetyl-CoA reductase and PHB polymerase were detected using polyclonal antibodies raised against these enzymes in rabbits and horse-radish peroxidase labeled secondary antibodies followed by chemiluminescent detection (USB/Amersham).

β-ketothiolase and NADP-specific acetoacetyl-CoA reductase activities were measured as described by Nishimura et al. (1978, Arch. Microbiol. 116: 21-24) and Saito et al. (1977, Arch. Microbiol. 114: 211-217) respectively. The acetoacetyl-CoA thiolase activity is measured as degradation of a Mg²⁺-acetoacetyl-CoA complex by monitoring the decrease in absorbance at 304 nm after addition of cell free extract using a Hewlett-Packer spectrophotometer. The acetoacetyl-CoA reductase activity is measured by monitoring the conversion of NADPH to NADP at 340 nm using a Hewlett-Packer spectrophotometer.

### Example 1

### Construction of Thiolase-Reductase Fusion Protein (Thredase)

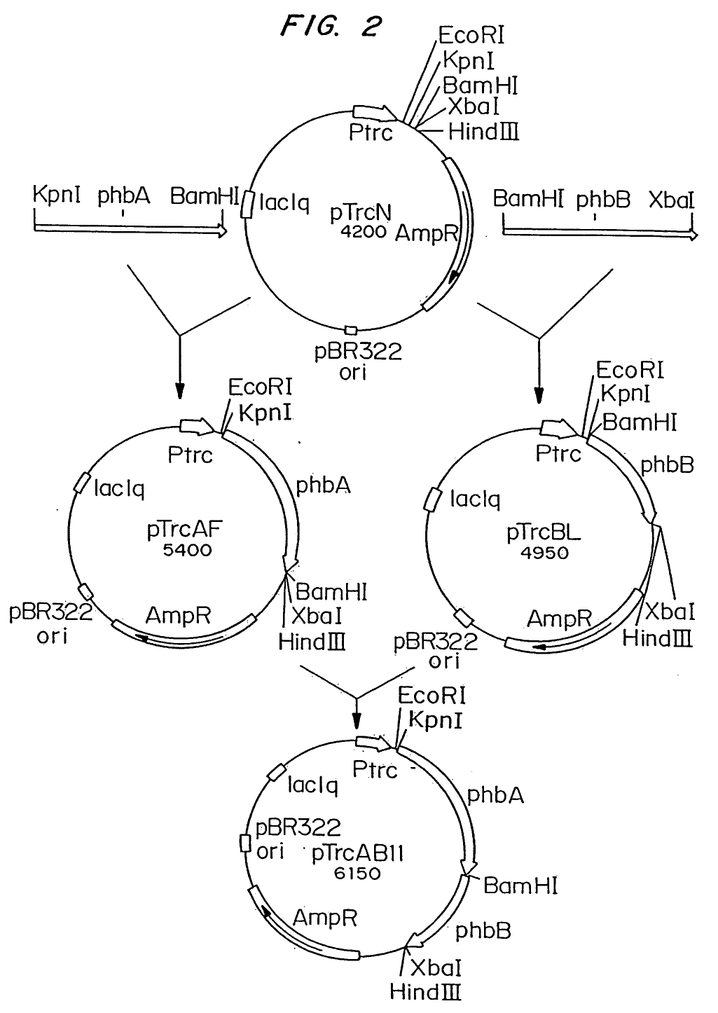

Plasmid pTrc AB11 was constructed using the following techniuqes essentially as illustrated in FIG. 2. The phbA gene from A. eutrophus was amplified from plasmid pAeT413, a derivative of plasmid pAeT41 (Peoples, O. P. and Sinskey, A. J., 1989, J. Biol. Chem. 264: 15298-15303): by thermal cycling (30 cycles of 40 sec. at 94° C., 40 sec. at 65° C. and 2 min at 72° C., followed by a final extension step at 72° C. for 7 min.) with the following primers. The DNA sequence and the amino acid sequence of phbA from A. eutrophus is shown in SEQ ID NO: 1 and SEQ ID NO: 2

A1FKpn

(SEQ ID NO: 3)

(GGGGTACCAGGAGGTTTTTATGACTGACGTTGTCATCGTATCC)

(SEQ ID NO: 4)

(CGCGGATCCTTTGCGCTCGACTGCCAGCGCCACGCCC).

A1F-Bam
A1F-Kpn contains the ribosome binding site and translational start site; A1F-Bam does not include the translational stop codon. The A. eutrophus phbB gene was amplified from a derivative of plasmid pAeT41 (Peoples, O. P. and Sinskey, A. J., 1989, J. Biol. Chem. 264: 15298-15303) by thermal cycling (30 cycles of 40 sec. at 94° C., 40 sec. at 45° C. and 2 min at 72° C., followed by a final extension step at 72° C. for 7 min.) with the following primers. The DNA sequence and the amino acid sequence of phbB from A. eutrophus is shown in SEQ ID NO: 5 and SEQ ID NO: 6.

B1L-Bam

(SEQ ID NO: 7)

(CGCGGATCCATGACTCAGCGCATTGCGTATGTGACC)

B1L-Xba

(SEQ ID NO: 8)

(GCTCTAGATCAGCCCATATGCAGGCCGCCGTTGAGCG).

B1L-Bam contains an ATG initiation codon next to the BamHI site but no translational intiation signals; B1L-Xba contains the translational stop codon TGA. The amplified phbA gene was then digested with KpnI and BamHI and the amplified phbB gene was digested with BamHI and XbaI. Following digestion, the phbA gene was cloned into pTrcN which had been digested with KpnI and BamHI to produce pTrcAF and the phbB gene was cloned into BamHI/XbaI-digested pTrcN to produce pTrcBL.

After confirmation of the DNA sequence of the insert, phbB was cloned as a BamHI/XbaI fragment from pTrcBL into BamHI/XbaI digested pTrcAF resulting in plasmid pTrcAB11. The resulting hybrid gene encodes for a thiolase-glycine-serine-reductase fusion. The DNA sequence and the amino acid sequence of the AB11 fusion is shown in SEQ ID NO: 9 and SEQ ID NO: 10.

The insertion of the BamHI site between phbA and phbB results in a glycine-serine linker that connects the thiolase and the reductase enzyme and which could be subsequently modified to alter the length and/or sequence of the linker region. Several such derivatives of pTrcAB11 were constructed as follows: pTrcAB11 was digested with BamHI and the linearized fragment purified and dephosphorylated with shrimp alkaline phosphatase.

Oligonucleotides were designed to insert the following DNA fragments into the BamHI site. The encoded amino acid sequence is indicated:

| | |
|---|---|
| L5A 5′ GATCTACCG 3′ | (SEQ ID NO: 11) |
| L5B 3′ ATGGCCTAG 5′ | (SEQ ID NO: 12) |
| G S T G S | (SEQ ID NO: 13) |

Oligonucleotides L5A and L5B (500 pmol) were phosphorylated using T4 polynucleotide kinase and annealed (133 pmol of each primer) and ligated into linearized pTrcAB11. The ligation mixture was electroporated into E. coli MBX240 and plasmids with the linker inserted between the thiolase and reductase genes were identified by restriction enzyme digestion with BsaWI.

The utility of the fusion constructs was investigated by transforming them into E. coli MBX240 and examining the integrity of the fusion at the polypeptide level by immunoblotting at the protein level by enzyme assays and for the production of PHB. MBX240 was derived from E. coli XL1-blue by integration of the A. eutrophus phaC gene (Peoples, O. P. and Sinskey, A. J., 1989, J. Biol. Chem. 264: 15298-15303). An alternative approach to the integrated strain would be to have expressed the PHB synthase from a compatible plasmid.

Recombinant strains containing the appropriate fusion plasmid were grown overnight in 2xYT/1% glucose/100 μg/ml ampicillin at 30 C. The grown culture was diluted 1:100 into 50 ml of fresh 2xYT/1% glucose/100 μg/ml ampicillin and incubated at 30 C. Two identical sets of cultures were inoculated, one which was induced with IPTG and one was not induced. Once the culture reached an OD₆₀₀ of 0.6, samples were induced with a final concentration of 1 mM IPTG. Cells were harvested 24 hours after induction by splitting into two 50 ml samples and centrifugation at 3000×g for 10 minutes. Samples of whole cells were retained for analysis of PHB content. The second set of pellets were resuspended in 0.75 ml of lysis buffer (50 mM Tris, 1 mM EDTA, 20% glycerol, pH 8.2) and sonicated (50% output, 2 min. at 50%). The crude extract was then centrifuged (10 min 3000×g, 4° C.) and the supernatant and pellet were separated on 10% SDS-PAGE gels and analyzed by Coomassie staining as well as by immuno-blotting. Immuno-blots were probed with rabbit anti-A. eutrophus thiolase and rabbit anti-A. eutrophus reductase antibodies. Both antibodies reacted with an Mr=62 kD protein which was absent from the control strain, MBX240 containing the vector pTrcN alone. There was no cross reactivity of the anti-thiolase antibodies with an Mr 42 kD polypeptide or of the reductase antibodies with an Mr 26 kD polypeptide. The soluble protein was then analyzed for thiolase and reductase activity.

The results of these analysis are presented in Table 1 for pTrcAB11 and five derivatives with modified linkers.

TABLE 1
Fusion Enzyme Activities

| | | thiolase | reductase | |
|---|---|---|---|---|
| fusion^{a} | induction^{b} | activity^{c} | activity^{c} | % PHB^{d} |
| pTrcN | − | 0.03 | 0.05 | 0 |
| | + | 0.03 | 0.03 | 0 |
| AB11 | − | 0.15 | 0.09 | 28.6 |
| | + | 0.32 | 0.07 | 56.3 |
| L5-1 | − | 0.44 | 0.08 | 32.4 |
| | + | 0.97 | 0.12 | 62.5 |
| L5-2 | − | 0.25 | 0.07 | 34.2 |
| | + | 0.37 | 0.09 | 57.6 |
| L5-3 | − | 0.38 | 0.06 | 40.4 |
| | + | 1.18 | 0.09 | 63.6 |
| L5-4 | − | 0.51 | 0.11 | 37.6 |
| | + | 2.21 | 0.17 | 65.3 |
| L5-5 | − | 0.44 | 0.11 | 36.0 |
| | + | 1.85 | 0.23 | 64.1 |

^{a}construct inserted in pTrcN, L5-n indicates an AB11 fusion with a linker derived from the L5 oligonucleotide set;
^{b}culture was induced (+) 1 mM IPTG at an OD600 for 24 hours or was uninduced (−);
^{c}thiolase and reductase activity in U/mg of crude protein extract;
^{d}accumulated PHB as percentage of the cell dry weight.

The results presented in Table 1 indicate that these thiolase-reductase fusions have both enzyme activities and result in the production of high levels of PHB.

The fusion encoded by pTrcAB11 was partially purified. A culture of E. coli MBX240 (XL1-Blue::phbC150) [pTrcAB11] cells grown at 16° C. for 33 hours (5.5 g) were resuspended in 11 ml of lysis buffer (50 mM Tris, 1 mM EDTA, 0.05% (w/v) Hecameg, 20% glycerol, pH 8.0) and sonicated (50% output, 2 min at 50%). The crude extract was then centrifuged (10 min 3000×g, 4° C.) and the supernatant was applied to a pre-equilibrated Toyopearl-DEAE 650S (Rohm & Haas, PA) column (16.5×3.0 cm) in 50 mM NaCl. Unbound protein was washed off with a 50 mM NaCl (300 ml) after which bound protein was eluted with a 50-500 mM NaCl gradient (400 ml total volume). Fractions containing both thiolase and reductase activity (eluted at 250 mM NaCl) were pooled and concentrated/desalted on a 50,000 MW spin column (Amicon). The active protein sample was further purified over a BLUE-SEPHAROSE™ CL6B (Pharmacia Biotech AB, Sweden) column (10.5 cm×2.6 cm) using the same buffers as for the DEAE but containing different NaCl concentrations. Unbound protein was washed off the column with 250 mM NaCl (200 ml) and the remaining protein was eluted in two steps using 750 mM NaCl and 2M NaCl. Two thirds of the thiolase and reductase activities were recovered in the 750 mM NaCl step with the remainder eluting in the 2M NaCl step. Again, fractions containing both thiolase and reductase activity were pooled and concentrated/desalted on a 50,000 MW spin column. The fusion protein preparation was analyzed by SDS-PAGE proteins detected by either Coomassie. Blue staining or Western-blot analysis using anti-β-ketothiolase and anti-acetoacetyl-CoA reductase antibodies. Fractions that contained both β-ketothiolase and acetoacetyl-CoA reductase activity showed a single protein band with an apparent molecular weight of 60 kDa that reacted with both antibodies, confirming both enzyme activities were present on a single polypeptide chain encoded by a single gene.

### Example 2

### Construction of Reductase-Thiolase Fusion Protein

A hybrid gene that expresses a reductase-glycine-serine-thiolase enzyme was constructed from PCR products containing the reductase and thiolase genes. The following primers

B1F-Kpn

(SEQ ID NO: 14)

(GGGGTACCAGGAGGTTTTTATGACTCAGCGCATTGCGTATGTGACC)

B1F-BamHI

(SEQ ID NO: 15)

(CGCGGATCCGCCCATATGCAGGCCGCCGTTGAGCG)

A1L-BamHI

(SEQ ID NO: 16)

(CGCGGATCCATGACTGACGTTGTCATCGTATCC)

A1L-XbaI

(SEQ ID NO: 17)

(GCTCTAGATTATTTGCGCTCGACTGCCAGCGCCACGCCC)

were used to amplify (30 cycles of 40 sec. at 94° C., 40 sec. at 65° C. and 2 min at 72° C., followed by a final extension step at 72° C. for 7 min.) these genes such that the reductase gene is preceded by a ribosome binding site and does not contain a stop codon. The stop codon of the fusion is provided by the thiolase gene.

The amplified phbB gene was digested with KpnI and BamHI, then cloned into the KpnI-BamHI site of pTrcN to produce pTrcBF. The amplified phbA gene was digested with BamHI and XbaI, and was cloned into the BamHI-XbaI site of pTrcN to obtain plasmid pTrcAL. The phbB gene from pTrcBF was digested with BamHI-KpnI and the fragment was inserted it into the BamHI-KpnI site of pTrcAL to obtain plasmid pTrcBA, resulting in a fusion gene coding for reductase-glycine-serine-thiolase in one polypeptide. The DNA sequence and the amino acid sequence of the B1A1 fusion is shown in SEQ ID NO: 18 and SEQ ID NO: 19.

### Example 3

### Design of PHA Synthase-ACP::CoA Transferase Fusions

The phaC1 gene encoding PHA synthase 1 of P. oleovorans (Huisman et. al., 1991, J. Biol. Chem. 266: 2191-2198) (C3) can be amplified by polymerase chain reaction using the following primers. The DNA sequence and the amino acid sequence of phbC1 gene of P. oleovorans is shown in SEQ ID NO: 20 and SEQ ID NO: 21.

C3 up I

(SEQ ID NO: 22)

5′ g-GAATTC-aggaggtttt-ATGAGTAACAAGAACAACGATGAGC 3′

C3 up II

(SEQ ID NO: 23)

5′ CG-GGATCC-acgctcgtgaacgtaggtgccc 3′

C3 dw I

(SEQ ID NO: 24)

5′ CG-GGATCC-AGTAACAAGAACAACGATGAGC 3′

C3 dw II

(SEQ ID NO: 25)

5′ GC-TCTAGA-AGCTT-TCAACGCTCGTGAACGTAGGTGCCC 3′

The phaG gene encoding acyl-ACP::CoA transferase from P. putida (G3) can be amplified by polymerase chain reaction using the following primers. The DNA sequence and the amino acid sequence of phaG gene of P. putida are shown in SEQ ID NO: 26 and SEQ ID NO: 27.

G3 dw I

(SEQ ID NO: 28)

5′ CG-GGATCC-AGGCCAGAAATCGCTGTACTTG 3′

G3 dw II

(SEQ ID NO: 29)

5′ GC-TCTAGA-AGCTT-TCAGATGGCAAATGCATGCTGCCCC 3′

G3 up I

(SEQ ID NO: 30)

5′ G-GAATTC-AGGAGGTTTT-ATGAGGCCAGAAATCGCTGTACTTG 3′

G3 up II

(SEQ ID NO: 31)

5′ CG-GGATCC-GATGGCAAATGCATGCTGCCCC 3′.

Fusions of C3 and G3 are subsequently created by cloning either the C3 up and G3 dw PCR products, or the G3 up and C3 dw PCR products as EcoRI-BamHI and BamHI-HindIII fragments into pTrcN. The resulting plasmids code for either a synthase-transferase fusion (C3G3) or transferase-synthase (G3C3) fusion protein. The DNA sequence and the amino acid sequence of C3G3 is shown in SEQ ID NO: 32 and SEQ ID NO: 33, and the DNA sequence and the amino acid sequence of G3C3 gene are shown in SEQ ID NO: 34 and SEQ ID, NO: 35.

### Example 4

### Design of PHA Synthase-Hydratase Fusions

The phaC gene encoding a PHB synthase fusion from Z. ramigera (C5) was amplified by polymerase chain reaction using the following primers. The DNA sequence and the amino acid sequence of phbC gene of Z. ramigera are shown in SEQ ID NO: 36 and SEQ ID NO: 37.

C5 up I

(SEQ ID NO: 38)

5′ G-GAGCTC-AGGAGGTTTT-ATGAGTAACAAGAACAACGATGAGC 3′

C5 up II

(SEQ ID NO: 39)

5′ CG-GGATCC-GCCCTTGGCTTTGACGTAACGG 3′

C5 dw I

(SEQ ID NO: 40)

5′ CG-GGATCC-AGTAACAAGAACAACGATGAGC 3′

C5 dw II

(SEQ ID NO: 41)

5′ GC-TCTAGA-AGCTT-TCAGCCCTTGGCTTTGACGTAACGG 3′

The phaJ gene encoding (R)-specific enoyl-CoA transferase from A. caviae (J12) can be amplified by polymerase chain reaction using the following primers. The DNA sequence and the amino acid sequence of phbJ gene of A. caviae are shown in SEQ ID NO: 42 and SEQ ID NO: 43.

J12 dw I

(SEQ ID NO: 44)

5′ CG-GGATCC-AGCGCACAATCCCTGGAAGTAG 3′

J12 dw II

(SEQ ID NO: 45)

5′ GC-TCTAGA-AGCTT-TTAAGGCAGCTTGACCACGGCTTCC 3′

J12 up I

(SEQ ID NO: 46)

5′ AG-GAGCTC-AGGAGGTTTT-ATGAGCGCACAATCCCTGGAAGTAG 3′

J12 up II

(SEQ ID NO: 47)

5′ CG-GGATCC-AGGCAGCTTGACCACGGCTTCC 3′

Fusions of C5 and J12 are subsequently created by cloning either the C5 up and J12 dw PCR products, or the J12 up and C5 dw PCR products as EcoRI-BamHI and BamHI-HindIII fragments into pTrcN. The resulting plasmids encode either a synthase-hydratase (C5J12) or hydratase-synthase (J12C5) fusion enzyme. The DNA sequence and the amino acid sequence of C5J12 RE shown in SEQ ID. NO: 48 and SEQ ID NO: 49, and the DNA sequence and the amino acid sequence of J12C5 gene are shown in SEQ ID NO: 50 and SEQ ID NO: 51.

### Example 5

### Design of Broad-Substrate Range Thiolase-Reductase Fusions

The bktB gene encoding thiolase II of R. eutropha (Slater et al. J. Bacteriol. (1998) 180, 1979-1987) (A1-II) can be amplified by polymerase chain reaction using the following primers. The DNA sequence and the amino acid sequence of bktB gene of R. eutropha are shown in SEQ ID NO: 52 and SEQ ID NO: 53.

A1-II up I

(SEQ ID NO: 54)

5′ G-GAATTC-AGGAGGTTTT-ATGACGCGTGAAGTGGTAGTGGTAAG 3′

A1-II up II

(SEQ ID NO: 55)

5′ CG-GGATCC-GATACGCTCGAAGATGGCGGC 3′

A1-II dw I

(SEQ ID NO: 56)

5′ CG-GGATCC-ACGCGTGAAGTGGTAGTGGTAAG 3′

A1-II dw II

(SEQ ID NO: 57)

5′ GC-TCTAGA-AGCTT-TCAGATACGCTCGAAGATGGCGGC 3′

The phaB gene encoding acyl-CoA reductase from R. eutropha (B1) is amplified by polymerase chain reaction using the primers described in Example 1. Fusions of A1-II and B1 are subsequently created by cloning either the A1-II up and B1 dw PCR products, or the B1 up and A1-II dw PCR products as EcoRI-BamHI and BamHI-HindIII fragments into pTrcN. The resulting plasmids encode either a thiolase-reductase (A1-IIB1) or reductase-thiolase (B1A1-II)) fusion enzyme. The DNA sequence and the amino acid sequence of A1-IIB1 is shown in SEQ ID NO: 58 and SEQ ID NO: 59, and the DNA sequence and the amino acid sequence of B1A1-II gene are shown in SEQ ID NO: 60 and SEQ ID NO: 61.

Modifications and variations of the present invention will be obvious to those of skill in the art from the foregoing detailed description. Such modifications and variations are intended to come within the scope of the following claims.

1-6. (canceled)

7. A gene encoding protein fusions having a formula selected from the group consisting of E1-Lₙ-E2 or E2-Lₙ-E1, wherein E1 and #E2 are expressed as catalytically active enzymes in plants which act on substrate in successive reactions in a polyhydroxyalkanoate biosynthetic pathway and wherein E1 and E2 are selected from the group consisting of β-ketothiolases, acyl-CoA reductases, polyhydroxyalkanoate synthases, poly(3-hydroxybutyrate) synthetases, phasin (phaP); phaP and phaC (1D); phaC, enoyl-CoA hydratases and beta-hydroxyacyl-ACP::coenzyme-A transferase, in which linker Lₙ is a peptide of n amino acids that links the carboxyl terminus of E1 to the amino terminus of E2 or the carboxyl terminus of E2 to the amino terminus of E1.

8. The gene of claim 7 encoding a fusion protein selected from the group consisting of beta-ketothiolase (phbA) and acyl-CoA reductase (phbB); phbB and phbA; PHA synthase (phaC) and and beta-hydroxyacyl-ACP::coenzyme-A transferase (phbG); phbG and phaC; phaC and enoyl-CoA hydratases (phaJ); and phaJ and phaC.

9. The gene of claim 7 wherein n in the linker is between zero and 50 amino acids.

10. The gene of claim 7 wherein the linker is glycine-serine.

11. The gene of claim 7 comprising a promoter for expression in plants.

12. The gene of claim 11 comprising a promoter specific for expression in a tissue, plastid or other organ.

13. The gene of claim 11 comprising a promoter specific for expression during a regulatory phase.

14. The gene of claim 7 further comprising RNA processing signals or ribozyme sequences.


SEQUENCE LISTING (61 sequences)

SEQ ID NO: 1 - DNA, 1182 bp, Alcaligenes eutrophus
  gene (1)..(1182): phbA gene
atgactgacgttgtcatcgtatccgccgcccgcaccgcggtcggcaagtttggcggctcgctggccaagatcccggcaccggaactgggtgccgtggtcatcaaggccgcgctggagcgcgccggcgtcaagccggagcaggtgagcgaagtcatcatgggccaggtgctgaccgccggttcgggccagaaccccgcacgccaggccgcgatcaaggccggcctgccggcgatggtgccggccatgaccatcaacaaggtgtgcggctcgggcctgaaggccgtgatgctggccgccaacgcgatcatggcgggcgacgccgagatcgtggtggccggcggccaggaaaacatgagcgccgccccgcacgtgctgccgggctcgcgcgatggtttccgcatgggcgatgccaagctggtcgacaccatgatcgtcgacggcctgtgggacgtgtacaaccagtaccacatgggcatcaccgccgagaacgtggccaaggaatacggcatcacacgcgaggcgcaggatgagttcgccgtcggctcgcagaacaaggccgaagccgcgcagaaggccggcaagtttgacgaagagatcgtcccggtgctgatcccgcagcgcaagggcgacccggtggccttcaagaccgacgagttcgtgcgccagggcgccacgctggacagcatgtccggcctcaagcccgccttcgacaaggccggcacggtgaccgcggccaacgcctcgggcctgaacgacggcgccgccgcggtggtggtgatgtcggcggccaaggccaaggaactgggcctgaccccgctggccacgatcaagagctatgccaacgccggtgtcgatcccaaggtgatgggcatgggcccggtgccggcctccaagcgcgccctgtcgcgcgccgagtggaccccgcaagacctggacctgatggagatcaacgaggcctttgccgcgcaggcgctggcggtgcaccagcagatgggctgggacacctccaaggtcaatgtgaacggcggcgccatcgccatcggccacccgatcggcgcgtcgggctgccgtatcctggtgacgctgctgcacgagatgaagcgccgtgacgcgaagaagggcctggcctcgctgtgcatcggcggcggcatgggcgtggcgctggcagtcgagcgcaaataa

SEQ ID NO: 2 - protein, 393 aa, Alcaligenes eutrophus
  PEPTIDE (1)..(393): beta-ketothiolase
MTDVVIVSAARTAVGKFGGSLAKIPAPELGAVVIKAALERAGVKPEQVSEVIMGQVLTAGSGQNPARQAAIKAGLPAMVPAMTINKVCGSGLKAVMLAANAIMAGDAEIVVAGGQENMSAAPHVLPGSRDGFRMGDAKLVDTMIVDGLWDVYNQYHMGITAENVAKEYGITREAQDEFAVGSQNKAEAAQKAGKFDEEIVPVLIPQRKGDPVAFKTDEFVRQGATLDSMSGLKPAFDKAGTVTAANASGLNDGAAAVVVMSAAKAKELGLTPLATIKSYANAGVDPKVMGMGPVPASKRALSRAEWTPQDLDLMEINEAFAAQALAVHQQMGWDTSKVNVNGGAIAIGHPIGASGCRILVTLLHEMKRRDAKKGLASLCIGGGMGVALAVERK

SEQ ID NO: 3 - DNA, 43 bp, Artificial Sequence
  Description of Artificial Sequence oligonucleotide primer- A1FKpn
ggggtaccaggaggtttttatgactgacgttgtcatcgtatcc

SEQ ID NO: 4 - DNA, 37 bp, Artificial Sequence
  Description of Artificial Sequence oligonucleotide primer- A1F-Bam
cgcggatcctttgcgctcgactgccagcgccacgccc

SEQ ID NO: 5 - DNA, 741 bp, Alcaligenes eutrophus
  gene (1)..(741): phbB gene
atgactcagcgcattgcgtatgtgaccggcggcatgggtggtatcggaaccgccatttgccagcggctggccaaggatggctttcgtgtggtggccggttgcggccccaactcgccgcgccgcgaaaagtggctggagcagcagaaggccctgggcttcgatttcattgcctcggaaggcaatgtggctgactgggactcgaccaagaccgcattcgacaaggtcaagtccgaggtcggcgaggttgatgtgctgatcaacaacgccggtatcacccgcgacgtggtgttccgcaagatgacccgcgccgactgggatgcggtgatcgacaccaacctgacctcgctgttcaacgtcaccaagcaggtgatcgacggcatggccgaccgtggctggggccgcatcgtcaacatctcgtcggtgaacgggcagaagggccagttcggccagaccaactactccaccgccaaggccggcctgcatggcttcaccatggcactggcgcaggaagtggcgaccaagggcgtgaccgtcaacacggtctctccgggctatatcgccaccgacatggtcaaggcgatccgccaggacgtgctcgacaagatcgtcgcgacgatcccggtcaagcgcctgggcctgccggaagagatcgcctcgatctgcgcctggttgtcgtcggaggagtccggtttctcgaccggcgccgacttctcgctcaacggcggcctgcatatgggctga

SEQ ID NO: 6 - protein, 246 aa, Alcaligenes eutrophus
  PEPTIDE (1)..(246): reductase
MTQRIAYVTGGMGGIGTAICQRLAKDGFRVVAGCGPNSPRREKWLEQQKALGFDFIASEGNVADWDSTKTAFDKVKSEVGEVDVLINNAGITRDVVFRKMTRADWDAVIDTNLTSLFNVTKQVIDGMADRGWGRIVNISSVNGQKGQFGQTNYSTAKAGLHGFTMALAQEVATKGVTVNTVSPGYIATDMVKAIRQDVLDKIVATIPVKRLGLPEEIASICAWLSSEESGFSTGADFSLNGGLHMG

SEQ ID NO: 7 - DNA, 36 bp, Artificial Sequence
  Description of Artificial Sequence oligonucleotide primer-B1L-Bam
cgcggatccatgactcagcgcattgcgtatgtgacc

SEQ ID NO: 8 - DNA, 37 bp, Artificial Sequence
  Description of Artificial Sequence oligonucleotide primer- B1L-Xba
gctctagatcagcccatatgcaggccgccgttgagcg

SEQ ID NO: 9 - DNA, 1926 bp, Alcaligenes eutrophus
  misc_feature (1)..(1926): phbA-linker-phbB fusion gene
atgactgacgttgtcatcgtatccgccgcccgcaccgcggtcggcaagtttggcggctcgctggccaagatcccggcaccggaactgggtgccgtggtcatcaaggccgcgctggagcgcgccggcgtcaagccggagcaggtgagcgaagtcatcatgggccaggtgctgaccgccggttcgggccagaaccccgcacgccaggccgcgatcaaggccggcctgccggcgatggtgccggccatgaccatcaacaaggtgtgcggctcgggcctgaaggccgtgatgctggccgccaacgcgatcatggcgggcgacgccgagatcgtggtggccggcggccaggaaaacatgagcgccgccccgcacgtgctgccgggctcgcgcgatggtttccgcatgggcgatgccaagctggtcgacaccatgatcgtcgacggcctgtgggacgtgtacaaccagtaccacatgggcatcaccgccgagaacgtggccaaggaatacggcatcacacgcgaggcgcaggatgagttcgccgtcggctcgcagaacaaggccgaagccgcgcagaaggccggcaagtttgacgaagagatcgtcccggtgctgatcccgcagcgcaagggcgacccggtggccttcaagaccgacgagttcgtgcgccagggcgccacgctggacagcatgtccggcctcaagcccgccttcgacaaggccggcacggtgaccgcggccaacgcctcgggcctgaacgacggcgccgccgcggtggtggtgatgtcggcggccaaggccaaggaactgggcctgaccccgctggccacgatcaagagctatgccaacgccggtgtcgatcccaaggtgatgggcatgggcccggtgccggcctccaagcgcgccctgtcgcgcgccgagtggaccccgcaagacctggacctgatggagatcaacgaggcctttgccgcgcaggcgctggcggtgcaccagcagatgggctgggacacctccaaggtcaatgtgaacggcggcgccatcgccatcggccacccgatcggcgcgtcgggctgccgtatcctggtgacgctgctgcacgagatgaagcgccgtgacgcgaagaagggcctggcctcgctgtgcatcggcggcggcatgggcgtggcgctggcagtcgagcgcaaaggatccatgactcagcgcattgcgtatgtgaccggcggcatgggtggtatcggaaccgccatttgccagcggctggccaaggatggctttcgtgtggtggccggttgcggccccaactcgccgcgccgcgaaaagtggctggagcagcagaaggccctgggcttcgatttcattgcctcggaaggcaatgtggctgactgggactcgaccaagaccgcattcgacaaggtcaagtccgaggtcggcgaggttgatgtgctgatcaacaacgccggtatcacccgcgacgtggtgttccgcaagatgacccgcgccgactgggatgcggtgatcgacaccaacctgacctcgctgttcaacgtcaccaagcaggtgatcgacggcatggccgaccgtggctggggccgcatcgtcaacatctcgtcggtgaacgggcagaagggccagttcggccagaccaactactccaccgccaaggccggcctgcatggcttcaccatggcactggcgcaggaagtggcgaccaagggcgtgaccgtcaacacggtctctccgggctatatcgccaccgacatggtcaaggcgatccgccaggacgtgctcgacaagatcgtcgcgacgatcccggtcaagcgcctgggcctgccggaagagatcgcctcgatctgcgcctggttgtcgtcggaggagtccggtttctcgaccggcgccgacttctcgctcaacggcggcctgcatatgggctga

SEQ ID NO: 10 - protein, 641 aa, Artificial Sequence
  Description of Artificial Sequence Thredase Fusion Protein
MTDVVIVSAARTAVGKFGGSLAKIPAPELGAVVIKAALERAGVKPEQVSEVIMGQVLTAGSGQNPARQAAIKAGLPAMVPAMTINKVCGSGLKAVMLAANAIMAGDAEIVVAGGQENMSAAPHVLPGSRDGFRMGDAKLVDTMIVDGLWDVYNQYHMGITAENVAKEYGITREAQDEFAVGSQNKAEAAQKAGKFDEEIVPVLIPQRKGDPVAFKTDEFVRQGATLDSMSGLKPAFDKAGTVTAANASGLNDGAAAVVVMSAAKAKELGLTPLATIKSYANAGVDPKVMGMGPVPASKRALSRAEWTPQDLDLMEINEAFAAQALAVHQQMGWDTSKVNVNGGAIAIGHPIGASGCRILVTLLHEMKRRDAKKGLASLCIGGGMGVALAVERKGSMTQRIAYVTGGMGGIGTAICQRLAKDGFRVVAGCGPNSPRREKWLEQQKALGFDFIASEGNVADWDSTKTAFDKVKSEVGEVDVLINNAGITRDVVFRKMTRADWDAVIDTNLTSLFNVTKQVIDGMADRGWGRIVNISSVNGQKGQFGQTNYSTAKAGLHGFTMALAQEVATKGVTVNTVSPGYIATDMVKAIRQDVLDKIVATIPVKRLGLPEEIASICAWLSSEESGFSTGADFSLNGGLHMG

SEQ ID NO: 11 - DNA, 9 bp, Artificial Sequence
  Description of Artificial Sequence oligonucleotide primer- L5A
gatctaccg

SEQ ID NO: 12 - DNA, 9 bp, Artificial Sequence
  Description of Artificial Sequence oligonucleotide primer- L5B
atggcctag

SEQ ID NO: 13 - protein, 5 aa, Artificial Sequence
  Description of Artificial Sequence Peptide Linker
GSTGS

SEQ ID NO: 14 - DNA, 46 bp, Artificial Sequence
  Description of Artificial Sequence oligonucleotide primer- B1F-Kpn
ggggtaccaggaggtttttatgactcagcgcattgcgtatgtgacc

SEQ ID NO: 15 - DNA, 35 bp, Artificial Sequence
  Description of Artificial Sequence oligonucleotide primer- B1F-BamHI
cgcggatccgcccatatgcaggccgccgttgagcg

SEQ ID NO: 16 - DNA, 33 bp, Artificial Sequence
  Description of Artificial Sequence oligonucleotide primer- A1L BamHI
cgcggatccatgactgacgttgtcatcgtatcc

SEQ ID NO: 17 - DNA, 39 bp, Artificial Sequence
  Description of Artificial Sequence oligonucleotide primer- A1L-XbaI
gctctagattatttgcgctcgactgccagcgccacgccc

SEQ ID NO: 18 - DNA, 1926 bp, Artificial Sequence
  gene (1)..(1926): phbB-linker-phbA fusion gene
atgactcagcgcattgcgtatgtgaccggcggcatgggtggtatcggaaccgccatttgccagcggctggccaaggatggctttcgtgtggtggccggttgcggccccaactcgccgcgccgcgaaaagtggctggagcagcagaaggccctgggcttcgatttcattgcctcggaaggcaatgtggctgactgggactcgaccaagaccgcattcgacaaggtcaagtccgaggtcggcgaggttgatgtgctgatcaacaacgccggtatcacccgcgacgtggtgttccgcaagatgacccgcgccgactgggatgcggtgatcgacaccaacctgacctcgctgttcaacgtcaccaagcaggtgatcgacggcatggccgaccgtggctggggccgcatcgtcaacatctcgtcggtgaacgggcagaagggccagttcggccagaccaactactccaccgccaaggccggcctgcatggcttcaccatggcactggcgcaggaagtggcgaccaagggcgtgaccgtcaacacggtctctccgggctatatcgccaccgacatggtcaaggcgatccgccaggacgtgctcgacaagatcgtcgcgacgatcccggtcaagcgcctgggcctgccggaagagatcgcctcgatctgcgcctggttgtcgtcggaggagtccggtttctcgaccggcgccgacttctcgctcaacggcggcctgcatatgggcggatccatgactgacgttgtcatcgtatccgccgcccgcaccgcggtcggcaagtttggcggctcgctggccaagatcccggcaccggaactgggtgccgtggtcatcaaggccgcgctggagcgcgccggcgtcaagccggagcaggtgagcgaagtcatcatgggccaggtgctgaccgccggttcgggccagaaccccgcacgccaggccgcgatcaaggccggcctgccggcgatggtgccggccatgaccatcaacaaggtgtgcggctcgggcctgaaggccgtgatgctggccgccaacgcgatcatggcgggcgacgccgagatcgtggtggccggcggccaggaaaacatgagcgccgccccgcacgtgctgccgggctcgcgcgatggtttccgcatgggcgatgccaagctggtcgacaccatgatcgtcgacggcctgtgggacgtgtacaaccagtaccacatgggcatcaccgccgagaacgtggccaaggaatacggcatcacacgcgaggcgcaggatgagttcgccgtcggctcgcagaacaaggccgaagccgcgcagaaggccggcaagtttgacgaagagatcgtcccggtgctgatcccgcagcgcaagggcgacccggtggccttcaagaccgacgagttcgtgcgccagggcgccacgctggacagcatgtccggcctcaagcccgccttcgacaaggccggcacggtgaccgcggccaacgcctcgggcctgaacgacggcgccgccgcggtggtggtgatgtcggcggccaaggccaaggaactgggcctgaccccgctggccacgatcaagagctatgccaacgccggtgtcgatcccaaggtgatgggcatgggcccggtgccggcctccaagcgcgccctgtcgcgcgccgagtggaccccgcaagacctggacctgatggagatcaacgaggcctttgccgcgcaggcgctggcggtgcaccagcagatgggctgggacacctccaaggtcaatgtgaacggcggcgccatcgccatcggccacccgatcggcgcgtcgggctgccgtatcctggtgacgctgctgcacgagatgaagcgccgtgacgcgaagaagggcctggcctcgctgtgcatcggcggcggcatgggcgtggcgctggcagtcgagcgcaaataa

SEQ ID NO: 19 - protein, 641 aa, Artificial Sequence
  Description of Artificial Sequence Reductase Fusion Protein
MTQRIAYVTGGMGGIGTAICQRLAKDGFRVVAGCGPNSPRREKWLEQQKALGFDFIASEGNVADWDSTKTAFDKVKSEVGEVDVLINNAGITRDVVFRKMTRADWDAVIDTNLTSLFNVTKQVIDGMADRGWGRIVNISSVNGQKGQFGQTNYSTAKAGLHGFTMALAQEVATKGVTVNTVSPGYIATDMVKAIRQDVLDKIVATIPVKRLGLPEEIASICAWLSSEESGFSTGADFSLNGGLHMGGSMTDVVIVSAARTAVGKFGGSLAKIPAPELGAVVIKAALERAGVKPEQVSEVIMGQVLTAGSGQNPARQAAIKAGLPAMVPAMTINKVCGSGLKAVMLAANAIMAGDAEIVVAGGQENMSAAPHVLPGSRDGFRMGDAKLVDTMIVDGLWDVYNQYHMGITAENVAKEYGITREAQDEFAVGSQNKAEAAQKAGKFDEEIVPVLIPQRKGDPVAFKTDEFVRQGATLDSMSGLKPAFDKAGTVTAANASGLNDGAAAVVVMSAAKAKELGLTPLATIKSYANAGVDPKVMGMGPVPASKRALSRAEWTPQDLDLMEINEAFAAQALAVHQQMGWDTSKVNVNGGAIAIGHPIGASGCRILVTLLHEMKRRDAKKGLASLCIGGGMGVALAVERK

SEQ ID NO: 20 - DNA, 1680 bp, Pseudomonas oleovorans
  gene (1)..(1680): phbC1 gene
atgagtaacaagaacaacgatgagctgcagcggcaggcctcggaaaacaccctggggctgaacccggtcatcggtatccgccgcaaagacctgttgagctcggcacgcaccgtgctgcgccaggccgtgcgccaaccgctgcacagcgccaagcatgtggcccactttggcctggagctgaagaacgtgctgctgggcaagtccagccttgccccggaaagcgacgaccgtcgcttcaatgacccggcatggagcaacaacccactttaccgccgctacctgcaaacctatctggcctggcgcaaggagctgcaggactggatcggcaacagcgacctgtcgccccaggacatcagccgcggccagttcgtcatcaacctgatgaccgaagccatggctccgaccaacaccctgtccaacccggcagcagtcaaacgcttcttcgaaaccggcggcaagagcctgctcgatggcctgtccaacctggccaaggacctggtcaacaacggtggcatgcccagccaggtgaacatggacgccttcgaggtgggcaagaacctgggcaccagtgaaggcgccgtggtgtaccgcaacgatgtgctggagctgatccagtacaagcccatcaccgagcaggtgcatgcccgcccgctgctggtggtgccgccgcagatcaacaagttctacgtattcgacctgagcccggaaaagagcctggcacgctactgcctgcgctcgcagcagcagaccttcatcatcagctggcgcaacccgaccaaagcccagcgcgaatggggcctgtccacctacatcgacgcgctcaaggaggcggtcgacgcggtgctggcgattaccggcagcaaggacctgaacatgctcggtgcctgctccggcggcatcacctgcacggcattggtcggccactatgccgccctcggcgaaaacaaggtcaatgccctgaccctgctggtcagcgtgctggacaccaccatggacaaccaggtcgccctgttcgtcgacgagcagactttggaggccgccaagcgccactcctaccaggccggtgtgctcgaaggcagcgagatggccaaggtgttcgcctggatgcgccccaacgacctgatctggaactactgggtcaacaactacctgctcggcaacgagccgccggtgttcgacatcctgttctggaacaacgacaccacgcgcctgccggccgccttccacggcgacctgatcgaaatgttcaagagcaacccgctgacccgcccggacgccctggaggtttgcggcactccgatcgacctgaaacaggtcaaatgcgacatctacagccttgccggcaccaacgaccacatcaccccgtggcagtcatgctaccgctcggcgcacctgttcggcggcaagatcgagttcgtgctgtccaacagcggccacatccagagcatcctcaacccgccaggcaaccccaaggcgcgcttcatgaccggtgccgatcgcccgggtgacccggtggcctggcaggaaaacgccaccaagcatgccgactcctggtggctgcactggcaaagctggctgggcgagcgtgccggcgagctggaaaaggcgccgacccgcctgggcaaccgtgcctatgccgctggcgaggcatccccgggcacctacgttcacgagcgttga

SEQ ID NO: 21 - protein, 559 aa, Pseudomonas oleovorans
  PEPTIDE (1)..(559): PHA Polymerase
MSNKNNDELQRQASENTLGLNPVIGIRRKDLLSSARTVLRQAVRQPLHSAKHVAHFGLELKNVLLGKSSLAPESDDRRFNDPAWSNNPLYRRYLQTYLAWRKELQDWIGNSDLSPQDISRGQFVINLMTEAMAPTNTLSNPAAVKRFFETGGKSLLDGLSNLAKDLVNNGGMPSQVNMDAFEVGKNLGTSEGAVVYRNDVLELIQYKPITEQVHARPLLVVPPQINKFYVFDLSPEKSLARYCLRSQQQTFIISWRNPTKAQREWGLSTYIDALKEAVDAVLAITGSKDLNMLGACSGGITCTALVGHYAALGENKVNALTLLVSVLDTTMDNQVALFVDEQTLEAAKRHSYQAGVLEGSEMAKVFAWMRPNDLIWNYWVNNYLLGNEPPVFDILFWNNDTTRLPAAFHGDLIEMFKSNPLTRPDALEVCGTPIDLKQVKCDIYSLAGTNDHITPWQSCYRSAHLFGGKIEFVLSNSGHIQSILNPPGNPKARFMTGADRPGDPVAWQENATKHADSWWLHWQSWLGERAGELEKAPTRLGNRAYAAGEASPGTYVHER

SEQ ID NO: 22 - DNA, 42 bp, Artificial Sequence
  Description of Artificial Sequence oligonucleotide primer- C3 up I
ggaattcaggaggttttatgagtaacaagaacaacgatgagc

SEQ ID NO: 23 - DNA, 30 bp, Artificial Sequence
  Description of Artificial Sequence oligonucleotide primer- C3 up II
cgggatccacgctcgtgaacgtaggtgccc

SEQ ID NO: 24 - DNA, 30 bp, Artificial Sequence
  Description of Artificial Sequence oligonucleotide primer- C3  dw I
cgggatccagtaacaagaacaacgatgagc

SEQ ID NO: 25 - DNA, 38 bp, Artificial Sequence
  Description of Artificial Sequence oligonucleotide primer- C3  dw II
gctctagaagctttcaacgctcgtgaacgtaggtgccc

SEQ ID NO: 26 - DNA, 888 bp, Pseudomonas putida
  gene (1)..(888): phaG
atgaggccagaaatcgctgtacttgatatccaaggtcagtatcgggtttacacggagttctatcgcgcggatgcggccgaaaacacgatcatcctgatcaacggctcgctggccaccacggcctcgttcgcccagacggtacgtaacctgcacccacagttcaacgtggttctgttcgaccagccgtattcaggcaagtccaagccgcacaaccgtcaggaacggctgatcagcaaggagaccgaggcgcatatcctccttgagctgatcgagcacttccaggcagaccacgtgatgtctttttcgtggggtggcgcaagcacgctgctggcgctggcgcaccagccgcggtacgtgaagaaggcagtggtgagttcgttctcgccagtgatcaacgagccgatgcgcgactatctggaccgtggctgccagtacctggccgcctgcgaccgttatcaggtcggcaacctggtcaatgacaccatcggcaagcacttgccgtcgctgttcaaacgcttcaactaccgccatgtgagcagcctggacagccacgagtacgcacagatgcacttccacatcaaccaggtgctggagcacgacctggaacgtgcgctgcaaggcgcgcgcaatatcaacatcccggtgctgttcatcaacggcgagcgcgacgagtacaccacagtcgaggatgcgcggcagttcagcaagcatgtgggcagaagccagttcagcgtgatccgcgatgcgggccacttcctggacatggagaacaagaccgcctgcgagaacacccgcaatgtcatgctgggcttcctcaagccaaccgtgcgtgaaccccgccaacgttaccaacccgtgcagcaggggcagcatgcatttgccatctga

SEQ ID NO: 27 - protein, 295 aa, Artificial Sequence
  Description of Artificial Sequence acyl ACP-CoA transferase
MRPEIAVLDIQGQYRVYTEFYRADAAENTIILINGSLATTASFAQTVRNLHPQFNVVLFDQPYSGKSKPHNRQERLISKETEAHILLELIEHFQADHVMSFSWGGASTLLALAHQPRYVKKAVVSSFSPVINEPMRDYLDRGCQYLAACDRYQVGNLVNDTIGKHLPSLFKRFNYRHVSSLDSHEYAQMHFHINQVLEHDLERALQGARNINIPVLFINGERDEYTTVEDARQFSKHVGRSQFSVIRDAGHFLDMENKTACENTRNVMLGFLKPTVREPRQRYQPVQQGQHAFAI

SEQ ID NO: 28 - DNA, 30 bp, Artificial Sequence
  Description of Artificial Sequence oligonucleotide primer- G3  dw I
cgggatccaggccagaaatcgctgtacttg

SEQ ID NO: 29 - DNA, 38 bp, Artificial Sequence
  Description of Artificial Sequence oligonucleotide primer- G3  dw II
gctctagaagctttcagatggcaaatgcatgctgcccc

SEQ ID NO: 30 - DNA, 42 bp, Artificial Sequence
  Description of Artificial Sequence oligonucleotide primer- G3  up I
ggaattcaggaggttttatgaggccagaaatcgctgtacttg

SEQ ID NO: 31 - DNA, 30 bp, Artificial Sequence
  Description of Artificial Sequence oligonucleotide primer- G3 up II
cgggatccgatggcaaatgcatgctgcccc

SEQ ID NO: 32 - DNA, 2571 bp, Pseudomonas putida
  gene (1)..(2571): phaC1-linker-phaG fusion gene
atgagtaacaagaacaacgatgagctgcagcggcaggcctcggaaaacaccctggggctgaacccggtcatcggtatccgccgcaaagacctgttgagctcggcacgcaccgtgctgcgccaggccgtgcgccaaccgctgcacagcgccaagcatgtggcccactttggcctggagctgaagaacgtgctgctgggcaagtccagccttgccccggaaagcgacgaccgtcgcttcaatgacccggcatggagcaacaacccactttaccgccgctacctgcaaacctatctggcctggcgcaaggagctgcaggactggatcggcaacagcgacctgtcgccccaggacatcagccgcggccagttcgtcatcaacctgatgaccgaagccatggctccgaccaacaccctgtccaacccggcagcagtcaaacgcttcttcgaaaccggcggcaagagcctgctcgatggcctgtccaacctggccaaggacctggtcaacaacggtggcatgcccagccaggtgaacatggacgccttcgaggtgggcaagaacctgggcaccagtgaaggcgccgtggtgtaccgcaacgatgtgctggagctgatccagtacaagcccatcaccgagcaggtgcatgcccgcccgctgctggtggtgccgccgcagatcaacaagttctacgtattcgacctgagcccggaaaagagcctggcacgctactgcctgcgctcgcagcagcagaccttcatcatcagctggcgcaacccgaccaaagcccagcgcgaatggggcctgtccacctacatcgacgcgctcaaggaggcggtcgacgcggtgctggcgattaccggcagcaaggacctgaacatgctcggtgcctgctccggcggcatcacctgcacggcattggtcggccactatgccgccctcggcgaaaacaaggtcaatgccctgaccctgctggtcagcgtgctggacaccaccatggacaaccaggtcgccctgttcgtcgacgagcagactttggaggccgccaagcgccactcctaccaggccggtgtgctcgaaggcagcgagatggccaaggtgttcgcctggatgcgccccaacgacctgatctggaactactgggtcaacaactacctgctcggcaacgagccgccggtgttcgacatcctgttctggaacaacgacaccacgcgcctgccggccgccttccacggcgacctgatcgaaatgttcaagagcaacccgctgacccgcccggacgccctggaggtttgcggcactccgatcgacctgaaacaggtcaaatgcgacatctacagccttgccggcaccaacgaccacatcaccccgtggcagtcatgctaccgctcggcgcacctgttcggcggcaagatcgagttcgtgctgtccaacagcggccacatccagagcatcctcaacccgccaggcaaccccaaggcgcgcttcatgaccggtgccgatcgcccgggtgacccggtggcctggcaggaaaacgccaccaagcatgccgactcctggtggctgcactggcaaagctggctgggcgagcgtgccggcgagctggaaaaggcgccgacccgcctgggcaaccgtgcctatgccgctggcgaggcatccccgggcacctacgttcacgagcgtggattcatgaggccagaaatcgctgtacttgatatccaaggtcagtatcgggtttacacggagttctatcgcgcggatgcggccgaaaacacgatcatcctgatcaacggctcgctggccaccacggcctcgttcgcccagacggtacgtaacctgcacccacagttcaacgtggttctgttcgaccagccgtattcaggcaagtccaagccgcacaaccgtcaggaacggctgatcagcaaggagaccgaggcgcatatcctccttgagctgatcgagcacttccaggcagaccacgtgatgtctttttcgtggggtggcgcaagcacgctgctggcgctggcgcaccagccgcggtacgtgaagaaggcagtggtgagttcgttctcgccagtgatcaacgagccgatgcgcgactatctggaccgtggctgccagtacctggccgcctgcgaccgttatcaggtcggcaacctggtcaatgacaccatcggcaagcacttgccgtcgctgttcaaacgcttcaactaccgccatgtgagcagcctggacagccacgagtacgcacagatgcacttccacatcaaccaggtgctggagcacgacctggaacgtgcgctgcaaggcgcgcgcaatatcaacatcccggtgctgttcatcaacggcgagcgcgacgagtacaccacagtcgaggatgcgcggcagttcagcaagcatgtgggcagaagccagttcagcgtgatccgcgatgcgggccacttcctggacatggagaacaagaccgcctgcgagaacacccgcaatgtcatgctgggcttcctcaagccaaccgtgcgtgaaccccgccaacgttaccaacccgtgcagcaggggcagcatgcatttgccatctga

SEQ ID NO: 33 - protein, 856 aa, Artificial Sequence
  Description of Artificial Sequence Synthase Acyl ACP-CoA Transferase Fusion Protein
MSNKNNDELQRQASENTLGLNPVIGIRRKDLLSSARTVLRQAVRQPLHSAKHVAHFGLELKNVLLGKSSLAPESDDRRFNDPAWSNNPLYRRYLQTYLAWRKELQDWIGNSDLSPQDISRGQFVINLMTEAMAPTNTLSNPAAVKRFFETGGKSLLDGLSNLAKDLVNNGGMPSQVNMDAFEVGKNLGTSEGAVVYRNDVLELIQYKPITEQVHARPLLVVPPQINKFYVFDLSPEKSLARYCLRSQQQTFIISWRNPTKAQREWGLSTYIDALKEAVDAVLAITGSKDLNMLGACSGGITCTALVGHYAALGENKVNALTLLVSVLDTTMDNQVALFVDEQTLEAAKRHSYQAGVLEGSEMAKVFAWMRPNDLIWNYWVNNYLLGNEPPVFDILFWNNDTTRLPAAFHGDLIEMFKSNPLTRPDALEVCGTPIDLKQVKCDIYSLAGTNDHITPWQSCYRSAHLFGGKIEFVLSNSGHIQSILNPPGNPKARFMTGADRPGDPVAWQENATKHADSWWLHWQSWLGERAGELEKAPTRLGNRAYAAGEASPGTYVHERGFMRPEIAVLDIQGQYRVYTEFYRADAAENTIILINGSLATTASFAQTVRNLHPQFNVVLFDQPYSGKSKPHNRQERLISKETEAHILLELIEHFQADHVMSFSWGGASTLLALAHQPRYVKKAVVSSFSPVINEPMRDYLDRGCQYLAACDRYQVGNLVNDTIGKHLPSLFKRFNYRHVSSLDSHEYAQMHFHINQVLEHDLERALQGARNINIPVLFINGERDEYTTVEDARQFSKHVGRSQFSVIRDAGHFLDMENKTACENTRNVMLGFLKPTVREPRQRYQPVQQGQHAFAI

SEQ ID NO: 34 - DNA, 2571 bp, Pseudomonas putida
  gene (1)..(2571): phaG-linker-phaC1 fusion gene
atgaggccagaaatcgctgtacttgatatccaaggtcagtatcgggtttacacggagttctatcgcgcggatgcggccgaaaacacgatcatcctgatcaacggctcgctggccaccacggcctcgttcgcccagacggtacgtaacctgcacccacagttcaacgtggttctgttcgaccagccgtattcaggcaagtccaagccgcacaaccgtcaggaacggctgatcagcaaggagaccgaggcgcatatcctccttgagctgatcgagcacttccaggcagaccacgtgatgtctttttcgtggggtggcgcaagcacgctgctggcgctggcgcaccagccgcggtacgtgaagaaggcagtggtgagttcgttctcgccagtgatcaacgagccgatgcgcgactatctggaccgtggctgccagtacctggccgcctgcgaccgttatcaggtcggcaacctggtcaatgacaccatcggcaagcacttgccgtcgctgttcaaacgcttcaactaccgccatgtgagcagcctggacagccacgagtacgcacagatgcacttccacatcaaccaggtgctggagcacgacctggaacgtgcgctgcaaggcgcgcgcaatatcaacatcccggtgctgttcatcaacggcgagcgcgacgagtacaccacagtcgaggatgcgcggcagttcagcaagcatgtgggcagaagccagttcagcgtgatccgcgatgcgggccacttcctggacatggagaacaagaccgcctgcgagaacacccgcaatgtcatgctgggcttcctcaagccaaccgtgcgtgaaccccgccaacgttaccaacccgtgcagcaggggcagcatgcatttgccatcggatccatgagtaacaagaacaacgatgagctgcagcggcaggcctcggaaaacaccctggggctgaacccggtcatcggtatccgccgcaaagacctgttgagctcggcacgcaccgtgctgcgccaggccgtgcgccaaccgctgcacagcgccaagcatgtggcccactttggcctggagctgaagaacgtgctgctgggcaagtccagccttgccccggaaagcgacgaccgtcgcttcaatgacccggcatggagcaacaacccactttaccgccgctacctgcaaacctatctggcctggcgcaaggagctgcaggactggatcggcaacagcgacctgtcgccccaggacatcagccgcggccagttcgtcatcaacctgatgaccgaagccatggctccgaccaacaccctgtccaacccggcagcagtcaaacgcttcttcgaaaccggcggcaagagcctgctcgatggcctgtccaacctggccaaggacctggtcaacaacggtggcatgcccagccaggtgaacatggacgccttcgaggtgggcaagaacctgggcaccagtgaaggcgccgtggtgtaccgcaacgatgtgctggagctgatccagtacaagcccatcaccgagcaggtgcatgcccgcccgctgctggtggtgccgccgcagatcaacaagttctacgtattcgacctgagcccggaaaagagcctggcacgctactgcctgcgctcgcagcagcagaccttcatcatcagctggcgcaacccgaccaaagcccagcgcgaatggggcctgtccacctacatcgacgcgctcaaggaggcggtcgacgcggtgctggcgattaccggcagcaaggacctgaacatgctcggtgcctgctccggcggcatcacctgcacggcattggtcggccactatgccgccctcggcgaaaacaaggtcaatgccctgaccctgctggtcagcgtgctggacaccaccatggacaaccaggtcgccctgttcgtcgacgagcagactttggaggccgccaagcgccactcctaccaggccggtgtgctcgaaggcagcgagatggccaaggtgttcgcctggatgcgccccaacgacctgatctggaactactgggtcaacaactacctgctcggcaacgagccgccggtgttcgacatcctgttctggaacaacgacaccacgcgcctgccggccgccttccacggcgacctgatcgaaatgttcaagagcaacccgctgacccgcccggacgccctggaggtttgcggcactccgatcgacctgaaacaggtcaaatgcgacatctacagccttgccggcaccaacgaccacatcaccccgtggcagtcatgctaccgctcggcgcacctgttcggcggcaagatcgagttcgtgctgtccaacagcggccacatccagagcatcctcaacccgccaggcaaccccaaggcgcgcttcatgaccggtgccgatcgcccgggtgacccggtggcctggcaggaaaacgccaccaagcatgccgactcctggtggctgcactggcaaagctggctgggcgagcgtgccggcgagctggaaaaggcgccgacccgcctgggcaaccgtgcctatgccgctggcgaggcatccccgggcacctacgttcacgagcgttga

SEQ ID NO: 35 - protein, 856 aa, Artificial Sequence
  Description of Artificial Sequence Acyl ACP-CoA Transferase Synthase Fusion Protein
MRPEIAVLDIQGQYRVYTEFYRADAAENTIILINGSLATTASFAQTVRNLHPQFNVVLFDQPYSGKSKPHNRQERLISKETEAHILLELIEHFQADHVMSFSWGGASTLLALAHQPRYVKKAVVSSFSPVINEPMRDYLDRGCQYLAACDRYQVGNLVNDTIGKHLPSLFKRFNYRHVSSLDSHEYAQMHFHINQVLEHDLERALQGARNINIPVLFINGERDEYTTVEDARQFSKHVGRSQFSVIRDAGHFLDMENKTACENTRNVMLGFLKPTVREPRQRYQPVQQGQHAFAIGSMSNKNNDELQRQASENTLGLNPVIGIRRKDLLSSARTVLRQAVRQPLHSAKHVAHFGLELKNVLLGKSSLAPESDDRRFNDPAWSNNPLYRRYLQTYLAWRKELQDWIGNSDLSPQDISRGQFVINLMTEAMAPTNTLSNPAAVKRFFETGGKSLLDGLSNLAKDLVNNGGMPSQVNMDAFEVGKNLGTSEGAVVYRNDVLELIQYKPITEQVHARPLLVVPPQINKFYVFDLSPEKSLARYCLRSQQQTFIISWRNPTKAQREWGLSTYIDALKEAVDAVLAITGSKDLNMLGACSGGITCTALVGHYAALGENKVNALTLLVSVLDTTMDNQVALFVDEQTLEAAKRHSYQAGVLEGSEMAKVFAWMRPNDLIWNYWVNNYLLGNEPPVFDILFWNNDTTRLPAAFHGDLIEMFKSNPLTRPDALEVCGTPIDLKQVKCDIYSLAGTNDHITPWQSCYRSAHLFGGKIEFVLSNSGHIQSILNPPGNPKARFMTGADRPGDPVAWQENATKHADSWWLHWQSWLGERAGELEKAPTRLGNRAYAAGEASPGTYVHER

SEQ ID NO: 36 - DNA, 1731 bp, Zoogloea ramigera
  gene (1)..(1731): phbC gene
atgaatttgcccgatccgcaagccattgccaacgcctggatgtcccaggtgggcgaccccagccaatggcaatcctggttcagcaaggcgcccaccaccgaggcgaacccgatggccaccatgttgcaggatatcggcgttgcgctcaaaccggaagcgatggagcagctgaaaaacgattatctgcgtgacttcaccgcgttgtggcaggattttttggctggcaaggcgccagccgtccagcgaccgcgcttcagctcggcagcctggcagggcaatccgatgtcggccttcaatgccgcatcttacctgctcaacgccaaattcctcagtgccatggtggaggcggtggacaccgcaccccagcaaaagcagaaaatacgctttgccgtgcagcaggtgattgatgccatgtcgcccgcgaacttcctcgccaccaacccggaagcgcagcaaaaactgattgaaaccaagggcgagagcctgacgcgtggcctggtcaatatgctgggcgatatcaatatgctgggcgatatcaacaacggccatatctcgctgtcggacgaatcggcctttgaagtgggccgcaacctggccattaccccgggcaccgtgatttacgaaaatccgctgttccagctgatccagtacacgccgaccacgccgacggtcagccagcgcccgctgttgatggtgccgccgtgcatcaacaagttctacatcctcgacctgcaaccggaaaattcgctggtgcgctacgcggtggagcagggcaacaccgtgttcctgatctcgtggagcaatccggacaagtcgctggccggcaccacctgggacgactacgtggagcagggcgtgatcgaagcgatccgcatcgtccaggacgtcagcggccaggacaagctgaacatgttcggcttctgcgtgggcggcaccatcgttgccaccgcactggcggtactggcggcgcgtggccagcacccggcggccagcctgaccctgctgaccaccttcctcgacttcagcgacaccgggtgctcgacgtcttgtcgagaaacccaggtcgcgctgcgtgaacagcaattgcgcgatggcggcctgatgccgggccgtgacctggcctcgaccttctcgagcctgcgtccgaacgacctggtatggaactatgtgcagtcgaactacctcaaaggcaatgagccggcggcgtttgacctgctgttctggaattcggacagcaccaatttgccgggcccgatgttctgctggtacctgcgcaacacctacctggaaaacagcctgaaagtgccgggcaagctgacggtggccggcgaaaagatcgacctcggcctgatcgacgccccggccttcatctacggttcgcgcgaagaccacatcgtgccgtggatgtcggcgtacggttcgctcgacatcctgaaccagggcaagccgggcgccaaccgcttcgtgctgggcgcgtccggccatatcgccggcgtgatcaactcggtggccaagaacaagcgcacgtactggatcaacgacggtggcgccgccgatgcccaggcctggttcgatggcgcgcaggaagtgccgggcagctggtggccgcaatgggccgggttcctgacccagcatggcggcaagaaggtcaagcccaaggccaagcccggcaacgcccgctacaccgcgatcgaggcggcgcccggccgttacgtcaaagccaagggctga

SEQ ID NO: 37 - protein, 576 aa, Zoogloea ramigera
  PEPTIDE (1)..(576): synthase
MNLPDPQAIANAWMSQVGDPSQWQSWFSKAPTTEANPMATMLQDIGVALKPEAMEQLKNDYLRDFTALWQDFLAGKAPAVQRPRFSSAAWQGNPMSAFNAASYLLNAKFLSAMVEAVDTAPQQKQKIRFAVQQVIDAMSPANFLATNPEAQQKLIETKGESLTRGLVNMLGDINMLGDINNGHISLSDESAFEVGRNLAITPGTVIYENPLFQLIQYTPTTPTVSQRPLLMVPPCINKFYILDLQPENSLVRYAVEQGNTVFLISWSNPDKSLAGTTWDDYVEQGVIEAIRIVQDVSGQDKLNMFGFCVGGTIVATALAVLAARGQHPAASLTLLTTFLDFSDTGCSTSCRETQVALREQQLRDGGLMPGRDLASTFSSLRPNDLVWNYVQSNYLKGNEPAAFDLLFWNSDSTNLPGPMFCWYLRNTYLENSLKVPGKLTVAGEKIDLGLIDAPAFIYGSREDHIVPWMSAYGSLDILNQGKPGANRFVLGASGHIAGVINSVAKNKRTYWINDGGAADAQAWFDGAQEVPGSWWPQWAGFLTQHGGKKVKPKAKPGNARYTAIEAAPGRYVKAKG

SEQ ID NO: 38 - DNA, 42 bp, Artificial Sequence
  Description of Artificial Sequence oligonucleotide primer- C5 up I
ggagctcaggaggttttatgagtaacaagaacaacgatgagc

SEQ ID NO: 39 - DNA, 30 bp, Artificial Sequence
  Description of Artificial Sequence oligonucleotide primer-C5 up II
cgggatccgcccttggctttgacgtaacgg

SEQ ID NO: 40 - DNA, 30 bp, Artificial Sequence; identical to SEQ ID NO: 24
  Description of Artificial Sequence oligonucleotide primer- C5 dw I

SEQ ID NO: 41 - DNA, 38 bp, Artificial Sequence
  Description of Artificial Sequence oligonucleotide primer- C5 dw II
gctctagaagctttcagcccttggctttgacgtaacgg

SEQ ID NO: 42 - DNA, 405 bp, Aeromonas caviae
  gene (1)..(405): phbJ gene
atgagcgcacaatccctggaagtaggccagaaggcccgtctcagcaagcggttcggggcggcggaggtagccgccttcgccgcgctctcggaggacttcaaccccctgcacctggacccggccttcgccgccaccacggcgttcgagcggcccatagtccacggcatgctgctcgccagcctcttctccgggctgctgggccagcagttgccgggcaaggggagcatctatctgggtcaaagcctcagcttcaagctgccggtctttgtcggggacgaggtgacggccgaggtggaggtgaccgcccttcgcgaggacaagcccatcgccaccctgaccacccgcatcttcacccaaggcggcgccctcgccgtgacgggggaagccgtggtcaagctgccttaa

SEQ ID NO: 43 - protein, 134 aa, Artificial Sequence
  Description of Artificial Sequence (R) specific enoyl-CoA transferase
MSAQSLEVGQKARLSKRFGAAEVAAFAALSEDFNPLHLDPAFAATTAFERPIVHGMLLASLFSGLLGQQLPGKGSIYLGQSLSFKLPVFVGDEVTAEVEVTALREDKPIATLTTRIFTQGGALAVTGEAVVKLP

SEQ ID NO: 44 - DNA, 30 bp, Artificial Sequence
  Description of Artificial Sequence oligonucleotide primer- J12 dw I
cgggatccagcgcacaatccctggaagtag

SEQ ID NO: 45 - DNA, 38 bp, Artificial Sequence
  Description of Artificial Sequence oligonucleotide primer-J12 dw II
gctctagaagcttttaaggcagcttgaccacggcttcc

SEQ ID NO: 46 - DNA, 43 bp, Artificial Sequence
  Description of Artificial Sequence J12 up I
aggagctcaggaggttttatgagcgcacaatccctggaagtag

SEQ ID NO: 47 - DNA, 30 bp, Artificial Sequence
  Description of Artificial Sequence oligonucleotide primer- J12 up II
cgggatccaggcagcttgaccacggcttcc

SEQ ID NO: 48 - DNA, 2139 bp, Artificial Sequence
  Description of Artificial Sequence Zoogloea ramigera and Aeromonas caviae phaC-linker-phbJ fusion gene
atgaatttgcccgatccgcaagccattgccaacgcctggatgtcccaggtgggcgaccccagccaatggcaatcctggttcagcaaggcgcccaccaccgaggcgaacccgatggccaccatgttgcaggatatcggcgttgcgctcaaaccggaagcgatggagcagctgaaaaacgattatctgcgtgacttcaccgcgttgtggcaggattttttggctggcaaggcgccagccgtccagcgaccgcgcttcagctcggcagcctggcagggcaatccgatgtcggccttcaatgccgcatcttacctgctcaacgccaaattcctcagtgccatggtggaggcggtggacaccgcaccccagcaaaagcagaaaatacgctttgccgtgcagcaggtgattgatgccatgtcgcccgcgaacttcctcgccaccaacccggaagcgcagcaaaaactgattgaaaccaagggcgagagcctgacgcgtggcctggtcaatatgctgggcgatatcaatatgctgggcgatatcaacaacggccatatctcgctgtcggacgaatcggcctttgaagtgggccgcaacctggccattaccccgggcaccgtgatttacgaaaatccgctgttccagctgatccagtacacgccgaccacgccgacggtcagccagcgcccgctgttgatggtgccgccgtgcatcaacaagttctacatcctcgacctgcaaccggaaaattcgctggtgcgctacgcggtggagcagggcaacaccgtgttcctgatctcgtggagcaatccggacaagtcgctggccggcaccacctgggacgactacgtggagcagggcgtgatcgaagcgatccgcatcgtccaggacgtcagcggccaggacaagctgaacatgttcggcttctgcgtgggcggcaccatcgttgccaccgcactggcggtactggcggcgcgtggccagcacccggcggccagcctgaccctgctgaccaccttcctcgacttcagcgacaccgggtgctcgacgtcttgtcgagaaacccaggtcgcgctgcgtgaacagcaattgcgcgatggcggcctgatgccgggccgtgacctggcctcgaccttctcgagcctgcgtccgaacgacctggtatggaactatgtgcagtcgaactacctcaaaggcaatgagccggcggcgtttgacctgctgttctggaattcggacagcaccaatttgccgggcccgatgttctgctggtacctgcgcaacacctacctggaaaacagcctgaaagtgccgggcaagctgacggtggccggcgaaaagatcgacctcggcctgatcgacgccccggccttcatctacggttcgcgcgaagaccacatcgtgccgtggatgtcggcgtacggttcgctcgacatcctgaaccagggcaagccgggcgccaaccgcttcgtgctgggcgcgtccggccatatcgccggcgtgatcaactcggtggccaagaacaagcgcacgtactggatcaacgacggtggcgccgccgatgcccaggcctggttcgatggcgcgcaggaagtgccgggcagctggtggccgcaatgggccgggttcctgacccagcatggcggcaagaaggtcaagcccaaggccaagcccggcaacgcccgctacaccgcgatcgaggcggcgcccggccgttacgtcaaagccaagggcggatccatgagcgcacaatccctggaagtaggccagaaggcccgtctcagcaagcggttcggggcggcggaggtagccgccttcgccgcgctctcggaggacttcaaccccctgcacctggacccggccttcgccgccaccacggcgttcgagcggcccatagtccacggcatgctgctcgccagcctcttctccgggctgctgggccagcagttgccgggcaaggggagcatctatctgggtcaaagcctcagcttcaagctgccggtctttgtcggggacgaggtgacggccgaggtggaggtgaccgcccttcgcgaggacaagcccatcgccaccctgaccacccgcatcttcacccaaggcggcgccctcgccgtgacgggggaagccgtggtcaagctgccttaa

SEQ ID NO: 49 - protein, 712 aa, Artificial Sequence
  Description of Artificial Sequence Synthase (R) specific enoyl-CoA transferase Fusion Protein
MNLPDPQAIANAWMSQVGDPSQWQSWFSKAPTTEANPMATMLQDIGVALKPEAMEQLKNDYLRDFTALWQDFLAGKAPAVQRPRFSSAAWQGNPMSAFNAASYLLNAKFLSAMVEAVDTAPQQKQKIRFAVQQVIDAMSPANFLATNPEAQQKLIETKGESLTRGLVNMLGDINMLGDINNGHISLSDESAFEVGRNLAITPGTVIYENPLFQLIQYTPTTPTVSQRPLLMVPPCINKFYILDLQPENSLVRYAVEQGNTVFLISWSNPDKSLAGTTWDDYVEQGVIEAIRIVQDVSGQDKLNMFGFCVGGTIVATALAVLAARGQHPAASLTLLTTFLDFSDTGCSTSCRETQVALREQQLRDGGLMPGRDLASTFSSLRPNDLVWNYVQSNYLKGNEPAAFDLLFWNSDSTNLPGPMFCWYLRNTYLENSLKVPGKLTVAGEKIDLGLIDAPAFIYGSREDHIVPWMSAYGSLDILNQGKPGANRFVLGASGHIAGVINSVAKNKRTYWINDGGAADAQAWFDGAQEVPGSWWPQWAGFLTQHGGKKVKPKAKPGNARYTAIEAAPGRYVKAKGGSMSAQSLEVGQKARLSKRFGAAEVAAFAALSEDFNPLHLDPAFAATTAFERPIVHGMLLASLFSGLLGQQLPGKGSIYLGQSLSFKLPVFVGDEVTAEVEVTALREDKPIATLTTRIFTQGGALAVTGEAVVKLP

SEQ ID NO: 50 - DNA, 2139 bp, Artificial Sequence
  Description of Artificial Sequence Aeromonas caviae and Zoogloea ramigera phbJ-linker-phaC fusion gene
atgagcgcacaatccctggaagtaggccagaaggcccgtctcagcaagcggttcggggcggcggaggtagccgccttcgccgcgctctcggaggacttcaaccccctgcacctggacccggccttcgccgccaccacggcgttcgagcggcccatagtccacggcatgctgctcgccagcctcttctccgggctgctgggccagcagttgccgggcaaggggagcatctatctgggtcaaagcctcagcttcaagctgccggtctttgtcggggacgaggtgacggccgaggtggaggtgaccgcccttcgcgaggacaagcccatcgccaccctgaccacccgcatcttcacccaaggcggcgccctcgccgtgacgggggaagccgtggtcaagctgcctggatccatgaatttgcccgatccgcaagccattgccaacgcctggatgtcccaggtgggcgaccccagccaatggcaatcctggttcagcaaggcgcccaccaccgaggcgaacccgatggccaccatgttgcaggatatcggcgttgcgctcaaaccggaagcgatggagcagctgaaaaacgattatctgcgtgacttcaccgcgttgtggcaggattttttggctggcaaggcgccagccgtccagcgaccgcgcttcagctcggcagcctggcagggcaatccgatgtcggccttcaatgccgcatcttacctgctcaacgccaaattcctcagtgccatggtggaggcggtggacaccgcaccccagcaaaagcagaaaatacgctttgccgtgcagcaggtgattgatgccatgtcgcccgcgaacttcctcgccaccaacccggaagcgcagcaaaaactgattgaaaccaagggcgagagcctgacgcgtggcctggtcaatatgctgggcgatatcaatatgctgggcgatatcaacaacggccatatctcgctgtcggacgaatcggcctttgaagtgggccgcaacctggccattaccccgggcaccgtgatttacgaaaatccgctgttccagctgatccagtacacgccgaccacgccgacggtcagccagcgcccgctgttgatggtgccgccgtgcatcaacaagttctacatcctcgacctgcaaccggaaaattcgctggtgcgctacgcggtggagcagggcaacaccgtgttcctgatctcgtggagcaatccggacaagtcgctggccggcaccacctgggacgactacgtggagcagggcgtgatcgaagcgatccgcatcgtccaggacgtcagcggccaggacaagctgaacatgttcggcttctgcgtgggcggcaccatcgttgccaccgcactggcggtactggcggcgcgtggccagcacccggcggccagcctgaccctgctgaccaccttcctcgacttcagcgacaccgggtgctcgacgtcttgtcgagaaacccaggtcgcgctgcgtgaacagcaattgcgcgatggcggcctgatgccgggccgtgacctggcctcgaccttctcgagcctgcgtccgaacgacctggtatggaactatgtgcagtcgaactacctcaaaggcaatgagccggcggcgtttgacctgctgttctggaattcggacagcaccaatttgccgggcccgatgttctgctggtacctgcgcaacacctacctggaaaacagcctgaaagtgccgggcaagctgacggtggccggcgaaaagatcgacctcggcctgatcgacgccccggccttcatctacggttcgcgcgaagaccacatcgtgccgtggatgtcggcgtacggttcgctcgacatcctgaaccagggcaagccgggcgccaaccgcttcgtgctgggcgcgtccggccatatcgccggcgtgatcaactcggtggccaagaacaagcgcacgtactggatcaacgacggtggcgccgccgatgcccaggcctggttcgatggcgcgcaggaagtgccgggcagctggtggccgcaatgggccgggttcctgacccagcatggcggcaagaaggtcaagcccaaggccaagcccggcaacgcccgctacaccgcgatcgaggcggcgcccggccgttacgtcaaagccaagggctga

SEQ ID NO: 51 - protein, 712 aa, Artificial Sequence
  Description of Artificial Sequence (R) - specific enoyl-CoA transferase Synthase Fusion Protein
MSAQSLEVGQKARLSKRFGAAEVAAFAALSEDFNPLHLDPAFAATTAFERPIVHGMLLASLFSGLLGQQLPGKGSIYLGQSLSFKLPVFVGDEVTAEVEVTALREDKPIATLTTRIFTQGGALAVTGEAVVKLPGSMNLPDPQAIANAWMSQVGDPSQWQSWFSKAPTTEANPMATMLQDIGVALKPEAMEQLKNDYLRDFTALWQDFLAGKAPAVQRPRFSSAAWQGNPMSAFNAASYLLNAKFLSAMVEAVDTAPQQKQKIRFAVQQVIDAMSPANFLATNPEAQQKLIETKGESLTRGLVNMLGDINMLGDINNGHISLSDESAFEVGRNLAITPGTVIYENPLFQLIQYTPTTPTVSQRPLLMVPPCINKFYILDLQPENSLVRYAVEQGNTVFLISWSNPDKSLAGTTWDDYVEQGVIEAIRIVQDVSGQDKLNMFGFCVGGTIVATALAVLAARGQHPAASLTLLTTFLDFSDTGCSTSCRETQVALREQQLRDGGLMPGRDLASTFSSLRPNDLVWNYVQSNYLKGNEPAAFDLLFWNSDSTNLPGPMFCWYLRNTYLENSLKVPGKLTVAGEKIDLGLIDAPAFIYGSREDHIVPWMSAYGSLDILNQGKPGANRFVLGASGHIAGVINSVAKNKRTYWINDGGAADAQAWFDGAQEVPGSWWPQWAGFLTQHGGKKVKPKAKPGNARYTAIEAAPGRYVKAKG

SEQ ID NO: 52 - DNA, 1185 bp, Aeromonas caviae
  gene (1)..(1185): bktB gene
atgacgcgtgaagtggtagtggtaagcggtgtccgtaccgcgatcgggacctttggcggcagcctgaaggatgtggcaccggcggagctgggcgcactggtggtgcgcgaggcgctggcgcgcgcgcaggtgtcgggcgacgatgtcggccacgtggtattcggcaacgtgatccagaccgagccgcgcgacatgtatctgggccgcgtcgcggccgtcaacggcggggtgacgatcaacgcccccgcgctgaccgtgaaccgcctgtgcggctcgggcctgcaggccattgtcagcgccgcgcagaccatcctgctgggcgataccgacgtcgccatcggcggcggcgcggaaagcatgagccgcgcaccgtacctggcgccggcagcgcgctggggcgcacgcatgggcgacgccggcctggtcgacatgatgctgggtgcgctgcacgatcccttccatcgcatccacatgggcgtgaccgccgagaatgtcgccaaggaatacgacatctcgcgcgcgcagcaggacgaggccgcgctggaatcgcaccgccgcgcttcggcagcgatcaaggccggctacttcaaggaccagatcgtcccggtggtgagcaagggccgcaagggcgacgtgaccttcgacaccgacgagcacgtgcgccatgacgccaccatcgacgacatgaccaagctcaggccggtcttcgtcaaggaaaacggcacggtcacggccggcaatgcctcgggcctgaacgacgccgccgccgcggtggtgatgatggagcgcgccgaagccgagcgccgcggcctgaagccgctggcccgcctggtgtcgtacggccatgccggcgtggacccgaaggccatgggcatcggcccggtgccggcgacgaagatcgcgctggagcgcgccggcctgcaggtgtcggacctggacgtgatcgaagccaacgaagcctttgccgcacaggcgtgcgccgtgaccaaggcgctcggtctggacccggccaaggttaacccgaacggctcgggcatctcgctgggccacccgatcggcgccaccggtgccctgatcacggtgaaggcgctgcatgagctgaaccgcgtgcagggccgctacgcgctggtgacgatgtgcatcggcggcgggcagggcattgccgccatcttcgagcgtatctga

SEQ ID NO: 53 - protein, 394 aa, Artificial Sequence
  Description of Artificial Sequence thiolase II
MTREVVVVSGVRTAIGTFGGSLKDVAPAELGALVVREALARAQVSGDDVGHVVFGNVIQTEPRDMYLGRVAAVNGGVTINAPALTVNRLCGSGLQAIVSAAQTILLGDTDVAIGGGAESMSRAPYLAPAARWGARMGDAGLVDMMLGALHDPFHRIHMGVTAENVAKEYDISRAQQDEAALESHRRASAAIKAGYFKDQIVPVVSKGRKGDVTFDTDEHVRHDATIDDMTKLRPVFVKENGTVTAGNASGLNDAAAAVVMMERAEAERRGLKPLARLVSYGHAGVDPKAMGIGPVPATKIALERAGLQVSDLDVIEANEAFAAQACAVTKALGLDPAKVNPNGSGISLGHPIGATGALITVKALHELNRVQGRYALVTMCIGGGQGIAAIFERI

SEQ ID NO: 54 - DNA, 43 bp, Artificial Sequence
  Description of Artificial Sequence oligonucleotide primer- A1 II up I
ggaattcaggaggttttatgacgcgtgaagtggtagtggtaag

SEQ ID NO: 55 - DNA, 29 bp, Artificial Sequence
  Description of Artificial Sequence oligonucleotide primer- A1-II up II
cgggatccgatacgctcgaagatggcggc

SEQ ID NO: 56 - DNA, 31 bp, Artificial Sequence
  Description of Artificial Sequence oligonucleotide primer- A1-II dw I
cgggatccacgcgtgaagtggtagtggtaag

SEQ ID NO: 57 - DNA, 37 bp, Artificial Sequence
  Description of Artificial Sequence oligonucleotide primer- A1-II dw II
gctctagaagctttcagatacgctcgaagatggcggc

SEQ ID NO: 58 - DNA, 1929 bp, Ralstonia eutropha
  gene (1)..(1929): bktB-linker-phbB fusion gene
atgacgcgtgaagtggtagtggtaagcggtgtccgtaccgcgatcgggacctttggcggcagcctgaaggatgtggcaccggcggagctgggcgcactggtggtgcgcgaggcgctggcgcgcgcgcaggtgtcgggcgacgatgtcggccacgtggtattcggcaacgtgatccagaccgagccgcgcgacatgtatctgggccgcgtcgcggccgtcaacggcggggtgacgatcaacgcccccgcgctgaccgtgaaccgcctgtgcggctcgggcctgcaggccattgtcagcgccgcgcagaccatcctgctgggcgataccgacgtcgccatcggcggcggcgcggaaagcatgagccgcgcaccgtacctggcgccggcagcgcgctggggcgcacgcatgggcgacgccggcctggtcgacatgatgctgggtgcgctgcacgatcccttccatcgcatccacatgggcgtgaccgccgagaatgtcgccaaggaatacgacatctcgcgcgcgcagcaggacgaggccgcgctggaatcgcaccgccgcgcttcggcagcgatcaaggccggctacttcaaggaccagatcgtcccggtggtgagcaagggccgcaagggcgacgtgaccttcgacaccgacgagcacgtgcgccatgacgccaccatcgacgacatgaccaagctcaggccggtcttcgtcaaggaaaacggcacggtcacggccggcaatgcctcgggcctgaacgacgccgccgccgcggtggtgatgatggagcgcgccgaagccgagcgccgcggcctgaagccgctggcccgcctggtgtcgtacggccatgccggcgtggacccgaaggccatgggcatcggcccggtgccggcgacgaagatcgcgctggagcgcgccggcctgcaggtgtcggacctggacgtgatcgaagccaacgaagcctttgccgcacaggcgtgcgccgtgaccaaggcgctcggtctggacccggccaaggttaacccgaacggctcgggcatctcgctgggccacccgatcggcgccaccggtgccctgatcacggtgaaggcgctgcatgagctgaaccgcgtgcagggccgctacgcgctggtgacgatgtgcatcggcggcgggcagggcattgccgccatcttcgagcgtatcggatccatgactcagcgcattgcgtatgtgaccggcggcatgggtggtatcggaaccgccatttgccagcggctggccaaggatggctttcgtgtggtggccggttgcggccccaactcgccgcgccgcgaaaagtggctggagcagcagaaggccctgggcttcgatttcattgcctcggaaggcaatgtggctgactgggactcgaccaagaccgcattcgacaaggtcaagtccgaggtcggcgaggttgatgtgctgatcaacaacgccggtatcacccgcgacgtggtgttccgcaagatgacccgcgccgactgggatgcggtgatcgacaccaacctgacctcgctgttcaacgtcaccaagcaggtgatcgacggcatggccgaccgtggctggggccgcatcgtcaacatctcgtcggtgaacgggcagaagggccagttcggccagaccaactactccaccgccaaggccggcctgcatggcttcaccatggcactggcgcaggaagtggcgaccaagggcgtgaccgtcaacacggtctctccgggctatatcgccaccgacatggtcaaggcgatccgccaggacgtgctcgacaagatcgtcgcgacgatcccggtcaagcgcctgggcctgccggaagagatcgcctcgatctgcgcctggttgtcgtcggaggagtccggtttctcgaccggcgccgacttctcgctcaacggcggcctgcatatgggctga

SEQ ID NO: 59 - protein, 642 aa, Artificial Sequence
  Description of Artificial Sequence Thiolase II Reductase Fusion Protein
MTREVVVVSGVRTAIGTFGGSLKDVAPAELGALVVREALARAQVSGDDVGHVVFGNVIQTEPRDMYLGRVAAVNGGVTINAPALTVNRLCGSGLQAIVSAAQTILLGDTDVAIGGGAESMSRAPYLAPAARWGARMGDAGLVDMMLGALHDPFHRIHMGVTAENVAKEYDISRAQQDEAALESHRRASAAIKAGYFKDQIVPVVSKGRKGDVTFDTDEHVRHDATIDDMTKLRPVFVKENGTVTAGNASGLNDAAAAVVMMERAEAERRGLKPLARLVSYGHAGVDPKAMGIGPVPATKIALERAGLQVSDLDVIEANEAFAAQACAVTKALGLDPAKVNPNGSGISLGHPIGATGALITVKALHELNRVQGRYALVTMCIGGGQGIAAIFERIGSMTQRIAYVTGGMGGIGTAICQRLAKDGFRVVAGCGPNSPRREKWLEQQKALGFDFIASEGNVADWDSTKTAFDKVKSEVGEVDVLINNAGITRDVVFRKMTRADWDAVIDTNLTSLFNVTKQVIDGMADRGWGRIVNISSVNGQKGQFGQTNYSTAKAGLHGFTMALAQEVATKGVTVNTVSPGYIATDMVKAIRQDVLDKIVATIPVKRLGLPEEIASICAWLSSEESGFSTGADFSLNGGLHMG

SEQ ID NO: 60 - DNA, 1929 bp, Ralstonia eutropha
  gene (1)..(1929): phbB-linker-bktB fusion gene
atgactcagcgcattgcgtatgtgaccggcggcatgggtggtatcggaaccgccatttgccagcggctggccaaggatggctttcgtgtggtggccggttgcggccccaactcgccgcgccgcgaaaagtggctggagcagcagaaggccctgggcttcgatttcattgcctcggaaggcaatgtggctgactgggactcgaccaagaccgcattcgacaaggtcaagtccgaggtcggcgaggttgatgtgctgatcaacaacgccggtatcacccgcgacgtggtgttccgcaagatgacccgcgccgactgggatgcggtgatcgacaccaacctgacctcgctgttcaacgtcaccaagcaggtgatcgacggcatggccgaccgtggctggggccgcatcgtcaacatctcgtcggtgaacgggcagaagggccagttcggccagaccaactactccaccgccaaggccggcctgcatggcttcaccatggcactggcgcaggaagtggcgaccaagggcgtgaccgtcaacacggtctctccgggctatatcgccaccgacatggtcaaggcgatccgccaggacgtgctcgacaagatcgtcgcgacgatcccggtcaagcgcctgggcctgccggaagagatcgcctcgatctgcgcctggttgtcgtcggaggagtccggtttctcgaccggcgccgacttctcgctcaacggcggcctgcatatgggcggatccatgacgcgtgaagtggtagtggtaagcggtgtccgtaccgcgatcgggacctttggcggcagcctgaaggatgtggcaccggcggagctgggcgcactggtggtgcgcgaggcgctggcgcgcgcgcaggtgtcgggcgacgatgtcggccacgtggtattcggcaacgtgatccagaccgagccgcgcgacatgtatctgggccgcgtcgcggccgtcaacggcggggtgacgatcaacgcccccgcgctgaccgtgaaccgcctgtgcggctcgggcctgcaggccattgtcagcgccgcgcagaccatcctgctgggcgataccgacgtcgccatcggcggcggcgcggaaagcatgagccgcgcaccgtacctggcgccggcagcgcgctggggcgcacgcatgggcgacgccggcctggtcgacatgatgctgggtgcgctgcacgatcccttccatcgcatccacatgggcgtgaccgccgagaatgtcgccaaggaatacgacatctcgcgcgcgcagcaggacgaggccgcgctggaatcgcaccgccgcgcttcggcagcgatcaaggccggctacttcaaggaccagatcgtcccggtggtgagcaagggccgcaagggcgacgtgaccttcgacaccgacgagcacgtgcgccatgacgccaccatcgacgacatgaccaagctcaggccggtcttcgtcaaggaaaacggcacggtcacggccggcaatgcctcgggcctgaacgacgccgccgccgcggtggtgatgatggagcgcgccgaagccgagcgccgcggcctgaagccgctggcccgcctggtgtcgtacggccatgccggcgtggacccgaaggccatgggcatcggcccggtgccggcgacgaagatcgcgctggagcgcgccggcctgcaggtgtcggacctggacgtgatcgaagccaacgaagcctttgccgcacaggcgtgcgccgtgaccaaggcgctcggtctggacccggccaaggttaacccgaacggctcgggcatctcgctgggccacccgatcggcgccaccggtgccctgatcacggtgaaggcgctgcatgagctgaaccgcgtgcagggccgctacgcgctggtgacgatgtgcatcggcggcgggcagggcattgccgccatcttcgagcgtatctga

SEQ ID NO: 61 - protein, 642 aa, Artificial Sequence
  Description of Artificial Sequence Reductase Thiolase II Fusion Protein
MTQRIAYVTGGMGGIGTAICQRLAKDGFRVVAGCGPNSPRREKWLEQQKALGFDFIASEGNVADWDSTKTAFDKVKSEVGEVDVLINNAGITRDVVFRKMTRADWDAVIDTNLTSLFNVTKQVIDGMADRGWGRIVNISSVNGQKGQFGQTNYSTAKAGLHGFTMALAQEVATKGVTVNTVSPGYIATDMVKAIRQDVLDKIVATIPVKRLGLPEEIASICAWLSSEESGFSTGADFSLNGGLHMGGSMTREVVVVSGVRTAIGTFGGSLKDVAPAELGALVVREALARAQVSGDDVGHVVFGNVIQTEPRDMYLGRVAAVNGGVTINAPALTVNRLCGSGLQAIVSAAQTILLGDTDVAIGGGAESMSRAPYLAPAARWGARMGDAGLVDMMLGALHDPFHRIHMGVTAENVAKEYDISRAQQDEAALESHRRASAAIKAGYFKDQIVPVVSKGRKGDVTFDTDEHVRHDATIDDMTKLRPVFVKENGTVTAGNASGLNDAAAAVVMMERAEAERRGLKPLARLVSYGHAGVDPKAMGIGPVPATKIALERAGLQVSDLDVIEANEAFAAQACAVTKALGLDPAKVNPNGSGISLGHPIGATGALITVKALHELNRVQGRYALVTMCIGGGQGIAAIFERI